(19) United States
(12) Patent Application Publication
(10) Pub. No.: US 2002/0032918 A1
(43) Pub. Date: Mar. 14, 2002

(54) Alteration of growth and adaptation under hypoxic conditions

(76) Inventors: Margret Maria Sauter, Hamburg (DE); Rene Lorbiecke, Hamburg (DE)

Correspondence Address: Ann R. Pokalsky, Esq., NIXON PEABODY LLP, 990 Stewart Avenue, Garden City, NY, 11530, US

(21) Appl. No.: 09/785,738

(22) Filed: Feb. 16, 2001

Related U.S. Application Data
(60) Provisional application No. 60/183,572, filed on Feb. 18, 2000.

(51) Int. Cl. C12N 15/29; A01H 5/00; C12P 21/02; C12N 15/82

(52) U.S. Cl. 800/289; 435/419; 435/468; 800/278; 435/320.1; 536/23.6; 530/370

(57) ABSTRACT

The present invention provides nucleotide sequences and corresponding amino acid sequences for a gene which confers the ability to adapt to low oxygen, i.e., hypoxic conditions. These sequences are referred to as SH2A and SH2A-like sequences. Genetic constructs and chimeric genes comprising these sequences are also provided. The nucleotide and amino acid sequences may be used to transform bacteria, yeasts, fungi, animal and plant species in order to modulate the level and/or activity of SH2A or SH2A-like protein. Also provided are transgenic plants, plant cells and host cells which express an SH2A or SHA2A-like gene. Methods for modulating growth or survival of cultured cells under hypoxic conditions, and methods for altering growth response in cells, tissues, or organs of an organism are also provided. In addition, methods for producing plants adapted to growth in hypoxic conditions, methods for improving water logging tolerance in a plant, methods for inducing gibberellin biosynthesis in a plant cell and methods of regulating an anaerobic response in a plant cell are provided by the present invention. The compositions and methodologies of the present invention find a myriad of uses in the horticultural, agricultural, medical, fermentation, and cell culture industries.

This application was originally filed as U.S. Provisional Application Ser. No. 06/183,572 on Feb. 18, 2000.

### BACKGROUND OF THE INVENTION

Deepwater rice (Oryza sativa L.) plants are specially adapted to withstand extended periods of partial submergence in water. Adaptation to low oxygen (hypoxic) conditions involves induction of anaerobiotic genes, some of which code for proteins which catalyze the fermentation pathway. Another adaptation found in several semiaquatic plants is the rapid growth of the youngest internode, which enables the plant to survive by keeping some of its leaves above the rising water. Accelerated growth of the internode is a result of increased cell division activity and enhanced cell elongation which has been well characterized in the literature (Kende, 1987; Kende et al., 1993; Lorbiecke and Sauter, 1998; Kende et al., 1998). The growth response is induced by an environmental signal and is mediated by the interaction of several plant hormones.

Submergence of plants leads to an increased ethylene concentration in plant tissue due to physical entrapment by the surrounding water and enhanced biosynthesis (Stunzi and Kende, 1989; Raskin and Kende, 1985). Ethylene alters the ratio of the growth promoting hormone gibberellic acid (GA) and cis-abscisic acid (ABA), a potent antagonist of GA action in rice internodes. Decreasing ABA levels and increasing GA levels lead to an increased responsiveness of the tissue to the GA present.

GA is the ultimate growth-promoting hormone in the internode (Raskin and Kende, 1984). Both application of ethylene and submergence reduces endogenous ABA levels in internodes by 75% within three hours (Hoffmann-Benning and Kende, 1992; Azuma et al., 1995). The endogenous GA₁ levels increase four-fold at the same time. The lag phase for the induction of growth by submergence is between three and four hours. (Hoffmann-Benning and Kende, 1992).

It has been shown genetically that a single gene locus is responsible for submergence tolerance in rice (Setter et al., 1997). Cloning and identification of this gene heretofore has been elusive. The present invention provides SH2 genes and corresponding SH2 proteins from different organisms. The subject SH2 genes are responsible for the induction of anaerobiosis-induced SH2 proteins and hence for one of the most basic mechanisms in adaptation to hypoxic conditions. Modulating the expression and/or activity of SH2 in a cell allows growth of the cell in conditions of low oxygen.

### SUMMARY OF THE INVENTION

The present invention provides transgenic plants, plant parts or plant cells which comprise a nucleotide sequence for an SH2A or SH2A-like gene wherein said nucleotide sequence is heterologous to the genome of said transgenic plant or plant cell.

Also provided are transgenic plants or plant cells which comprises a nucleotide sequence for an SH2A or SH2A-like gene wherein said nucleotide sequence has been introduced into the plant, plant part or plant cell by recombinant DNA means. Such plants may have an endogenous SH2A-like gene and using recombinant DNA methodologies, additional endogenous SH2A-like genes may be added to the plant, plant part or plant cell.

Also provided are transgenic plants, plant parts or plant cells comprising an SH2A or SH2A-like protein wherein said SH2A or SH2A-like protein is heterologous to the plant, plant part or plant cell.

Host cells comprising a nucleotide sequence for an SH2A or SH2A-like gene wherein said nucleotide sequence is heterologous to the genome of said host cell or wherein said nucleotide sequence has been introduced into said host cell by recombinant DNA means. Examples of host cells include bacterial, yeast, fungal, insect, plant or animal cells. Within the host cell, a nucleotide sequence for an SH2A or SH2A-like gene may be in the sense or antisense orientation relative to a regulatory region directing expression of said nucleotide sequence.

Methods for modulating growth or survival of cultured cells under hypoxic conditions which comprise modulating the level or activity of an SH2A or SH2A-like protein in said cultured cells are also provided. The present invention further provides methods for altering growth response in cultured cells by modulating the level or activity of an SH2A or SH2A-like protein in said cultured cells. Cultures cells may include bacterial, yeast, fungal, plant, mammalian or insect cells.

The present invention further provides a method for altering growth response in cells, tissues or organs of an organism which comprises modulating the level or activity of an SH2A or SH2A-like protein in said cells, tissues or organs of said organism. A method for altering growth response in cells, tissues or organs of a plant which comprises modulating the level or activity of an SH2A or SH2A-like protein in said cells, tissues or organs of said plant is also provided. For example, the level of SH2A or SH2A-like protein may be modulated by increasing transcription of a nucleotide sequence for said SH2A or SH2A-like protein. In plants, an increase in transcription is induced by exposing the cells, tissues or organs of a plant to ethephon or ethylene.

A method for producing plants adapted to growth in hypoxic conditions which comprises transforming at least one of a plant cell, pollen, protoplast, explant, plant part or plant organ with a coding sequence for an SH2A or like gene and regenerating a plant therefrom is also provided. Also provided is a method for improving survival of a plant in conditions of low oxygen which comprises transforming at least one of a plant cell, pollen, protoplast, explant, plant part or plant organ with a coding sequence for an SH2A or SH2A-like gene and regenerating a plant therefrom.

A method for improving water logging tolerance in a plant which comprises transforming at least one of a plant cell, pollen, protoplast, explant, plant part or plant organ with a coding sequence for an SH2A or SH2A-like gene and regenerating a plant therefrom is also provided.

The present invention also provides a method for inducing gibberellin biosynthesis in a plant cell, protoplast, explant, plant part or plant organ. The method comprises modulating the level or activity of SH2A or SH2A-like protein in said plant cell or protoplast. Similarly, a method for inducing gibberellin biosynthesis in a plant, said method comprising modulating the level or activity of SH2A or SH2A-like protein in the cells of said plant is provided by the present invention.

A method of regulating an anaerobic response protein in a plant cell, protoplast, explant, plant part or plant organ which comprises modulating the level or activity of an SH2A or SH2A-like protein therein is also provided. For example, the anaerobic response protein pyruvate decarboxylase 2 may be regulated in this manner.

The present invention also provides genetic constructs comprising a nucleotide sequence for an SH2A or SH2A-like gene operably linked to a promoter sequence which directs expression of said nucleotide sequence. The SH2A or SH2A-like gene may be e.g., a cDNA or genomic sequence. Genetic constructs may have the nucleotide sequence for an SH2A or SH2A-like gene contained therein in a sense or antisense orientation relative to the promoter sequence. Genetic constructs aimed at silencing expression of an SH2A or SH2A-like gene may have the nucleotide sequence for an SH2A or SH2A-like gene (or one or more fragments thereof) contained therein in a sense and/or antisense orientation relative to the promoter sequence.

Chimeric gene constructs comprising an SH2A or SH2A-like gene promoter operably linked to a heterologous coding sequence are also provided.

Isolated nucleic acids coding for an SH2A-like protein selected from the group consisting of nucleic acid sequences set forth in SEQ ID NOs:5, 7, 9, 11, 13, 15, and 17 are also provided by the present invention.

In addition, the present invention provides isolated SH2A-like proteins having an amino acid sequence selected from the group consisting of amino acid sequences set forth in SEQ ID NO:6, 8, 10, 12, 14, 16 and 18.

### BRIEF DESCRIPTION OF THE DRAWINGS

FIG. 1A is a Southern blot analysis of SH2A in Deepwater rice. Genomic DNA was digested with HindIII (H), BamHI (B), ClaI (C), PstI (P) or KpnI (K), blotted, and probed with a 3′-specific probe of SH2A under stringent conditions. The position of molecular length standards is indicated at left.

FIG. 1B is a Southern blot analysis of SH2B in Deepwater rice. The blot of FIG. 1A was washed and probed with a 3′-specific probe of SH2B under stringent conditions.

FIG. 1C is a Southern blot analysis of SH2-related sequences in Deepwater rice. To determine the copy number of SH2-related sequences the same blot depicted in FIGS. 1A and 1B was washed and then probed with the full-length cDNA of SH2A under low stringency conditions.

FIG. 2 is an alignment of deduced amino acid sequences from plant, animal, fungi and bacteria SH2-homologs. Amino acids that are conserved in 50% or more of the sequences are shaded. Dashes indicate gaps inserted to optimise alignment. A conserved proline residue in all analysed sequences is indicated by [*]. Incomplete sequences are indicated by [#]. For the mmsR protein of Pseudomonas aeruginosa only aa 1-164 are shown. For sequences indicated by [+], identified ESTs represent various regions of SH2-homologs without overlapping sequences.

FIG. 3 is a table indicating relationships between pairs of SH2 homolog amino acid sequences. Each sequence was compared to every other sequence using the genedoc program. The first number describes the percentage of identical residues between the two sequences. The number in parentheses indicates the percentage of similar and conservative substitutions between the two sequences. The shaded upper left quarter indicates sequences having 50% or greater sequence indentation.

FIG. 4 illustrates the localization, hydropathic analysis and alignment of the amino acid sequences in the homologous regions between SH2A and the mmsR protein of Pseudomonas aeruginosa. The position of a putative nuclear localization signal [NLS] and a putative destruction box motif is shown in the schematic drawing of the SH2A protein.

FIG. 5 is a dendrogram generated by the ClustalX program from an alignment of putative full-length SH2 homologs based on amino acid sequence comparison. The two SH2 homologs from rice are in bold text.

FIG. 6A is a Northern blot analysis of SH2A, SH2B and pyruvate decarboxylase 2 gene expression in tissues of partially submerged deepwater rice plants. Deepwater rice plants were submerged for up to 18 h. At the time points indicated, total RNA was isolated from the adventitious roots, the intercalary meristem, the elongation zone and the differentiation zone and analysed for expression of SH2A, SH2B and pyruvate decarboxylase 2.

FIG. 6B is a Northern blot analysis of Deepwater rice plant SH2A and pyruvate decarboxylase 2 gene expression in tissues of partially submerged deepwater rice plants. Plants were submerged for up to 6 h and expression of SH2A and pyruvate decarboxylase 2 was analysed in the intercalary meristem and in the elongation zone.

FIG. 7 is a Northern blot analysis of SH2A and pyruvate decarboxylase 2 gene expression in the intercalary meristem and in the elongation zone of isolated stem segments containing the youngest internode after incubation with ethephon (E), GA_{3(GA)} or NBD. After isolation of total RNA, SH2A and pyruvate decarboxylase 2 expression was analysed in the intercalary meristem and in the elongation zone. The ethidium bromide stained RNA gel is shown as a loading control.

FIG. 8A is a Northern blot analysis of SH2A and pyruvate decarboxylase 2 gene expression in the growing zone of isolated stem segments treated with 150 μM ethephon for up to 6 h. Stems without incubation (C₀) or stems incubated for 6 h in water (C₆) were used as controls. Total RNA was isolated at the indicated time points and gene expression of SH2A and pyruvate decarboxylase 2 was analysed.

FIG. 8B is a Northern blot analysis of SH2A and pyruvate decarboxylase 2 gene expression in the intercalary meristem of isolated stem segments treated with different concentrations of ethephon. Stem segments were incubated in beakers without (C) or with indicated concentrations of ethephon for 2.5 h. Total RNA was isolated and gene expression of SH2A and pyruvate decarboxylase 2 was analysed.

FIG. 9A is a Northern blot analysis of SH2A and pyruvate decarboxylase 2 gene expression in the intercalary meristem of isolated stem segments treated with or without 150 μM ethephon in the presence of the protein synthesis inhibitor cycloheximide (CHX). Stem segments were incubated with aqueous solutions of CHX at the indicated concentrations for 3 h.

FIG. 9B is a Northern blot analysis of SH2A and pyruvate decarboxylase 2 gene expression in the intercalary meristem of isolated stem segments treated with different concentrations of CHX for 3 h. Control segments (C) were incubated for 3 h in water.

FIG. 10 is a schematic drawing of cis-elements in the promoter regions of three SH2A-like genes from Arabidopsis thaliana.

### DETAILED DESCRIPTION OF THE INVENTION

In accordance with the present invention, it has been discovered that a gene from rice (Oryza sativa), designated SH2A, is involved in the ability of the plant to adapt to low oxygen, i.e, hypoxic, conditions. In addition, it has also been discovered that the gene product of SH2A belongs to a family of highly conserved proteins which occur ubiquitously in eukaryotes.

Hypoxic conditions associated with submergence are difficult to define as they are the result of flooding conditions, boundary layer effects and plant tissue metabolism. In most environments, floodwater oxygen concentrations are usually below air saturation (i.e., hypoxic). Especially during the night, however, oxygen can be completely absent, i.e., anoxic rather than hypoxic conditions. Even in floodwater saturated with oxygen, anoxic cores can still occur in submerged plant tissues (Setter et al. 1997 and references cited therein). As used herein, “hypoxic conditions” means any condition comprising anoxic conditions and any condition of oxygen subsaturation, i.e., relative to the air. In most instances, the anoxic or subsaturation conditions are temporary.

The present invention provides an SH2A gene and corresponding SH2A protein from rice. The present invention also provides SH2A-like genes and SH2A homologs from various organisms. As used herein, the terms “SH2A-like gene” and “SH2A or like gene” refer to a gene from an organism other than rice (Oryza sativa) which corresponds to the SH2A gene in rice as exhibited by homologous nucleotide sequence. The terms “SH2A-like protein” and “SH2A homolog” refer to a protein homologous to SH2A in an organism other than rice as exhibited by homologous amino acid sequence and the function of conferring adaptation and/or growth under hypoxic conditions. Thus the present invention is directed to SH2A and its derivatives, homologs and functional analogs. Use herein of the term “SH2A or like protein” encompasses all such homologous or heterologous derivatives, homologs, and functional analogs. The SH2A and SH2A-like genetic sequence and corresponding protein may be homologous to a particular cell, i.e., is naturally occurring in such cell or may be heterologous to the cell, i.e., the genetic sequences or protein may be introduced into the cell from a source not originating with the same organism.

In another aspect of the invention, there is provided a method for modulating growth or survival of cells under hypoxic conditions. The method comprises modulating the level and/or activity of an SH2A or SH2A-like protein in cells, tissues or organs of an organism. Such a modulation of the level and/or activity of SH2A or like protein allows adaptation of those cells, tissues or organs of the organism to conditions of hypoxia. In one embodiment, the organism is an animal. Particularly preferred are cells, tissues or organs of organisms belonging to a mammalian species. An SH2A homolog or SH2A agonist may be administered to animal cells in culture in order to modulate growth and survival of cells under hypoxic conditions. An SH2A homolog or SH2A agonist may also be administered to cells in vivo and at the site of hypoxia. Thus, a stroke or heart attack patient may be administered an effective amount of SH2A homolog or SH2A agonist in an amount sufficient to reduce the clinical effects of hypoxia. In an ex vivo gene therapy approach, an SH2A-like gene is used to transform human cells to over express an SH2A homolog and the transformed cells are transplanted into a patient, preferably at the site of hypoxia. Said method for modulation of the level and/or activity of an SH2A or SH2A-like protein in cells, tissues or organs of an animal, preferably mammal, may also be useful in curing or attenuating other hypoxia-related pathologies. Such hypoxic conditions in animals, preferably mammals, can be found and include end-stage renal disease characterized by progressive fibrosis (Norman et al. 1999), myocardial ischemia (Sinusas 1999), liver cirrhosis (Maruyama et al. 1994), high-altitude retinopathy and other altitude-related illnesses (Wiedman and Tabin 1999). Asphyxiated newborns are especially vulnerable to hypoxia and especially to hypoxic ischemic brain damage (Gucuyener et al. 1999) Hypoxic conditions in animals, preferably mammals, can also be found in the microenvironments created by tumor invasion, metastasis and lethality. Solid tumors are characterized by neovascularization and increased glycolysis as well as with the overexpression of a hypoxia-inducible transcription factor (Zhong et al. 1999).

In another embodiment, the current invention comprises a method for modulation of the level and/or activity of SH2A or SH2a-like proteins in tumor cells. Said method includes administration of e.g. a dominant-negative SH2A homolog or administration of e.g. an SH2A agonist to animal, preferably mammalian cells, in culture in order to decrease growth and survival of cells under hypoxic conditions. An SH2A homolog or SH2A agonist may also be administered to cells in vivo and at the site of hypoxia. Thus, a cancer patient may be administered an effective amount of SH2A homolog or SH2A agonist in an amount sufficient to reduce the growth of the cancerous tumor cells. In an ex vivo gene therapy approach, an SH2A-like gene, e.g. encoding a dominant-negative SH2A-like protein, is used to transform human cells to overexpress an SH2A homolog and the transformed cells are transplanted into a cancer patient, preferably at the site of tumor development. Preferably, the mammal is a human and the SH2A-like gene and corresponding gene product has the nucleotide and amino acid sequences set forth in SEQ ID NO: 13 and 14, respectively. Methods of transforming human cells, and promoter sequences which function in human cells are well known in the literature.

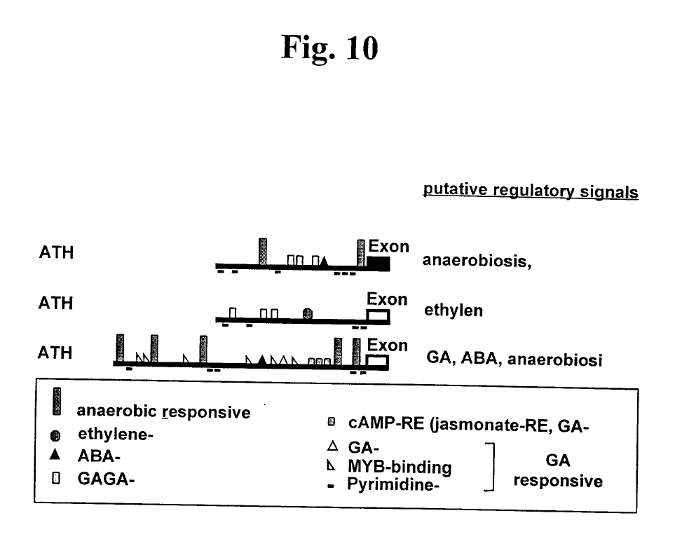

To overcome limited gas diffusion associated with flooding, rice plants can, depending on the cultivar, respond in either one of two ways: becoming tolerant to submergence or elongating in order to escape submergence. Elongation under flash flood conditions is a disadvantage because the taller plants tend to lodge once the water level recedes. Elongation is, however, desirable in areas where rice is grown in or floating on deepwater. Survival of seedlings of a submergence intolerant cultivar can be greatly enhanced when its elongation response is inhibited by spraying with paclobutrazol, a gibberellin biosynthesis inhibitor. Submergence tolerance is accompanied by efficient alcoholic fermentation as energy-producing mechanism (Setter et al. 1997 and references cited therein). Submergence conditions not only lead to hypoxia but also result in the accumulation of ethylene which in turn alters the ratio of the plant hormones GA and ABA. Analysis of the promoter regions of the SH2A-like genes of Arabidopsis thaliana (see FIG. 10/Example 7) shows that promoter elements are present responsive to one or more of the alterations (hypoxia, ethylene, GA, ABA) resulting from submergence.

Adaption to hypoxia and overcoming the adverse effects of hypoxia, either via development of submergence tolerance or via the elongation response, will clearly influence the grain yield. Moreover, an increased seed size, and thus an increased carbohydrate content, is an important factor in submergence tolerance of rice seedlings (Setter et al. 1997).

In another aspect of the invention, there is provided a method of altering growth response in cells, tissues or organs of a plant which comprises modulating the level and/or activity of an SH2A or like protein therein. Modulating the level or activity of SH2A or like protein allows alteration of growth of the cells, tissues or organs with respect to plant size and/or structure and/or yield. Modulating the level or activity of SH2A or SH2A homolog can be performed at the gene level, i.e., by transforming a plant with an SH2A or like gene or with a gene encoding a ribozyme targeted to the RNA of said SH2A or like gene. The SH2A or SH2A-like gene may be used to transform a plant cell in either a sense or antisense orientation. In order to effect changes in cell growth, e.g., an increase or decrease in elongation of a plant stem, an SH2A or like gene in the sense orientation is used to transform plant cells, protoplasts, explants, plant parts (e.g., embryo, ovary, pollen, roots, portions of the stem, hypocotyl, meristem etc.) or plant organs. An SH2A or SH2A-like gene may be used in sense orientation to result in cosuppression or in an antisense orientation to transform a plant cell, protoplast, explant, plant part or plant organ so that a plant regenerated therefrom shows e.g., an increase or a decrease in elongation of a plant stem. A similar effect can be obtained in plants regenerated from a plant cell, protoplast, explant, plant part or plant organ transformed with a gene encoding a ribozyme targeted to the RNA of the SH2A- or SH2A-like gene. Modulating the level or activity of SH2A or a SH2A homolog can furthermore be performed at the gene level by mutagenesis, e.g. by T-DNA or transposon insertion or by gene silencing strategies as described e.g. in WO 98/36083, WO 98/53083, WO 99/15682 or WO 99/53050. Genetic constructs aimed at silencing expression of an SH2A or SH2A-like gene may have the nucleotide sequence for an SH2A or SH2A-like gene (or one or more fragments thereof) contained therein in a sense and/or antisense orientation relative to the promoter sequence.

Modulating the level or activity of SH2A or SH2A like protein may also be performed at the protein level, by administering to cells or exposing cells to SH2A, to an SH2A homolog or to an SH2A agonist. Such an application finds particular use e.g., in cell and tissue culture of animal and plant cells. Modulating the level or activity of SH2A or SH2A-like proteins may also be mediated by immunomodulation, i.e. the expression of antibodies specific for the SH2A or SH2A-like protein in the host cell. Such antibodies comprise ‘plantibodies’, single chain antibodies, IgG antibodies, Fab fragments and heavy chain camel antibodies.

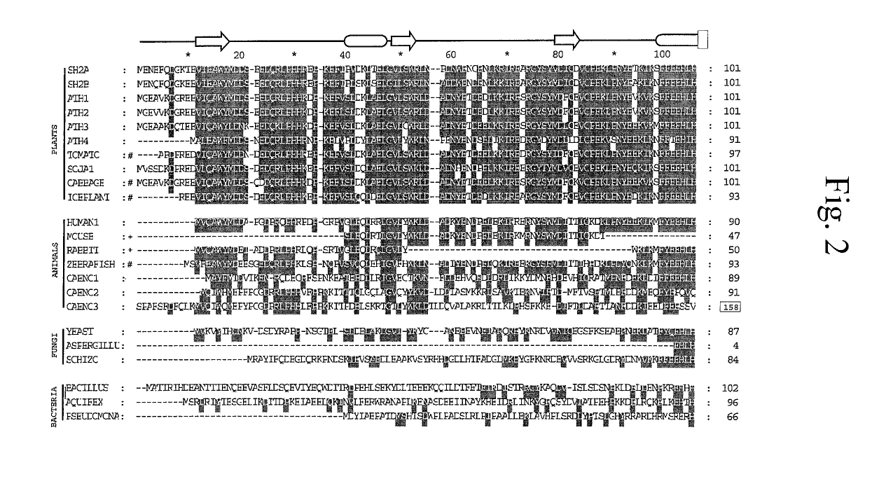

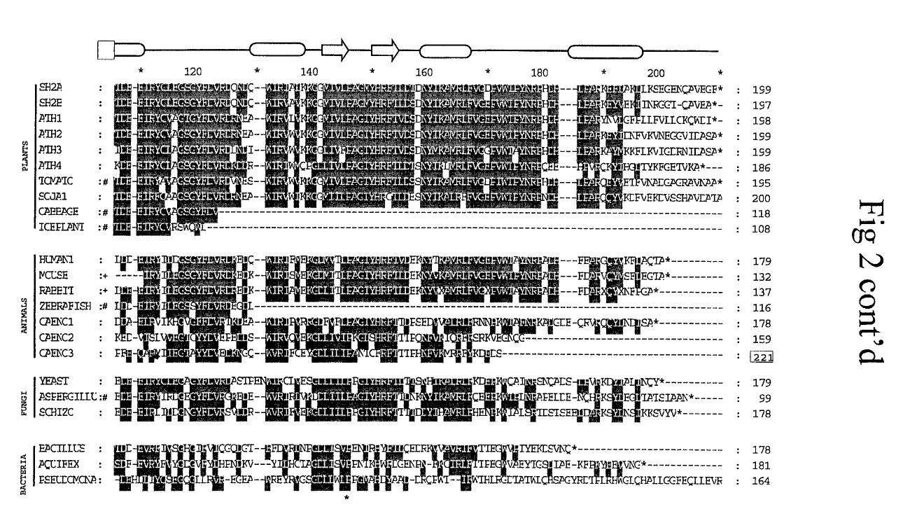

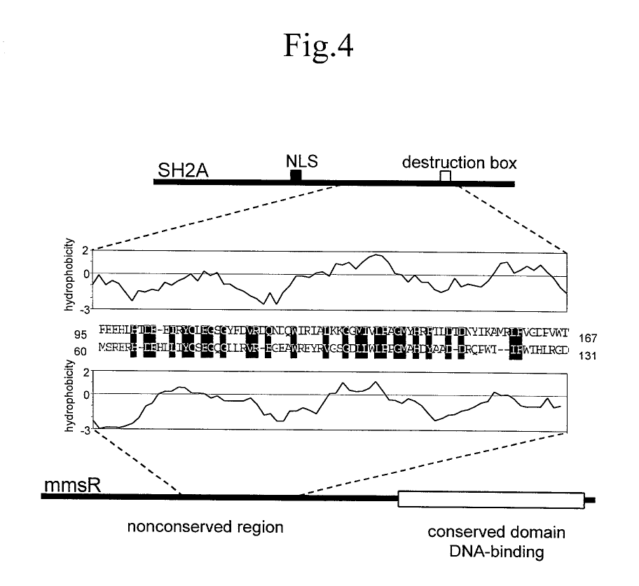

A closer examination of the SH2A and SH2A-like protein sequences reveals that most (see FIGS. 2 and 4) contain a nuclear localization sequence (NLS; KRXR, residues 64-67 in rice SH2A) and some a destruction box motif (RX₂LX₂,₃N, residues 145-152 in rice SH2A). NLSs are characteristic of proteins normally resident in the cytoplasm that translocate to and function in the nucleus under the influence of specific signals such as mitogens or stress. Such proteins comprise transcription factors as well as cell cycle proteins. Other proteins containing NLSs can act as a shuttle to transport proteins to and required in the nucleus but themselves not containing a NLS, i.e. the so-called piggy-back mechanism. The presence of a destruction box motif in a (usually regulatory) protein is indicative of the rapid turnover of such a protein which is an efficient mechanism to restrict the activity of said protein in time. It is to be expected that SH2A and SH2A-like proteins function in the nucleus and are thus involved in (a) nuclear process(es). Such processes include regulation of gene transcription (e.g. directly as a transcription factor or indirectly as shuttle for a transcription factor) and regulation of the cell cycle. In ammals, hypoxia modulates the cell cycle, i.e. hypoxia induces a growth suppressive state in mitogen-activated cells by inhibiting the synthesis of mitotic cyclins A and B (Naldini and Carraro 1999). Hypoxia also increases the levels of p21 (waf1/cip1), an inhibitor of cyclin-dependent kinases (Zaman et al. 1999). As many of the animal cell cycle proteins have their counterpart(s) in plants (see Mironov et al. 1999 for review on plant cell cycle), it is likely that hypoxia suppresses the plant cell cycle in submergence tolerant plants but enhances the plant cell cycle in the elongation response of submerged plants. It can thus not be excluded that SH2A or SH2A-like proteins function as regulators of gene transcription and/or regulators of the cell cycle. A potential role of SH2A or SH2A-like proteins as a regulator of the cell cycle can be envisaged as follows. As discussed supra for rice, plants can respond to submergence-induced hypoxia in either of two ways: by displaying tolerance or by displaying elongation (i.e. submergence susceptibility).

In a first model, the SH2A or SH2A-like proteins could act as negative regulators of cell cycle control genes or cell cycle control proteins. In submergence tolerant plants, a hypoxia-induced signalling cascade would lead to enhanced expression of SH2A or SH2A-like genes. As a result, the cell cycle would be blocked and no elongation would occur. In submergence susceptible plants displaying elongation, the absence of a key component of said hypoxia-induced signalling cascade would prevent accumulation of SH2A or SH2A-like proteins. As a result, the cell cycle would not be blocked and could subsequently be enhanced, e.g. due to reduced SH2A or SH2A-like mRNA stability or due to dilution of the SH2A or SH2A-like proteins during initial growth, resulting in a fast elongation response.

In a second model, the SH2A or SH2A-like proteins could act as positive regulators of cell cycle control genes or cell cycle control proteins. Said cell cycle control genes or cell cycle control proteins would in this case comprise, e.g. inhibitors of other cell cycle control genes or other cell cycle control proteins. The different plant responses, submergence tolerance or elongation can again be explained by the presence or absence, respectively, of a key component in the hypoxia-induced signalling cascade. In either of the two models, said key component of the hypoxia-induced signalling cascade could be encoded by e.g. a gene of the rice major locus for submergence tolerance (Xu and Mackill 1996).

The term “cell cycle” means the cyclic biochemical and structural events associated with growth and with division of cells, and in particular with the regulation of the replication of DNA and mitosis. Cell cycle includes phases called: G0, Gap1 (G1), DNA synthesis (S), Gap2 (G2), and mitosis (M). Normally these four phases occur sequentially, however, the cell cycle also includes modified cycles such as endomitosis, acytokinesis, polyploidy, polyteny, and endoreduplication.

The term “cell cycle interacting protein”, “cell cycle protein” or “cell cycle control protein” as denoted herein means a protein which exerts control on or regulates or is required for the cell cycle or part thereof of a cell, tissue, organ or whole organism and/or DNA replication. It may also be capable of binding to, regulating or being regulated by cyclin dependent kinases or their subunits. The term also includes peptides, polypeptides, fragments, variants, homologs, alleles or precursors (e.g. preproproteins) thereof.

Cell cycle control proteins and their role in regulating the cell cycle of eukaryotic organisms are reviewed in detail by John (1981) and the contributing papers therein (Norbury and Nurse 1992; Nurse 1990; Ormrod and Francis 1993) and the contributing papers therein (Doerner et al. 1996; Elledge 1996; Francis and Halford 1995; Francis et al. 1998; Hirt et al. 1991; Mironov et al. 1999) which are incorporated by reference.

The term “cell cycle control genes” refers to any gene or mutant thereof which exerts control on or are required for: chromosomal DNA synthesis and for mitosis (preprophase band, nuclear envelope, spindle formation, chromosome condensation, chromosome segregation, formation of new nuclei, formation of phragmoplast, etc.) meiosis, cytokinesis, cell growth, and endoreduplication. Cell cycle control genes are also all genes exerting control on the above such as homologues of CDKs, cyclins, E2Fs, Rv, CKI, Cks, and also any genes which interfere with the above such as e.g., cyclin D, cdc25, Wee1, Nim1, MAP kinases, etc.

More specifically, cell cycle control genes are all genes involved in the control of entry and progression through S phase. They include, not exclusively, genes expressing “cell cycle control proteins” such as cyclin dependent kinases (CDK), cyclin dependent kinase inhibitors (CKI), D, E and A cyclins, E2F and DP transcription factors, pocket proteins, CDC7/DBF4 kinase, CDC6, MCM2-7, Orc proteins, cdc45, components of SCF ubiquitin ligase, PCNA, DNA-polymerase.

The term “cell cycle control protein” includes cyclins A, B, C, D and E including CYCA1;1, CYCAC2;1, CYCA3;1, CYCB1;1, CYCB1;2, CYC B2;2, CYCD1;1, CYCD2;1, CYCD3;1, and CYCD4;1 (Evans et al. 1983; Francis et al. 1998; Labbe et al. 1989; Murray and Kirschner 1989; Renaudin et al. 1996; Soni et al. 1995; Sorrell et al. 1999; Swenson et al. 1986) cyclin dependent kinase inhibitor (CKI) proteins such as ICK1 (Wang et al. 1997), FL39, FL66, FL67 (PCT/EP98/05895), Sic1, Far1, Rum1, p21, p27, p57, p16, p15, p18, p19 (Elledge 1996; Pines 1995), p14 and p14ARF; p13suc1 or CKS1At (De Veylder et al. 1997; Hayles and Nurse 1986) and nim-1 (Russell and Nurse 1987a; Russell and Nurse 1987b; Fantes 1989; Russell and Nurse 1986; Russell and Nurse 1987a; Russell and Nurse 1987b) homologues of Cdc2 such as Cdc2MsB (Hirt et al. 1993) CdcMs kinase (Bogre et al. 1997) cdc2 T14Y15 phosphatases such as Cdc25 protein phosphatase or p80cdc25 (Bell et al. 1993; Elledge 1996; Kumagai and Dunphy 1991; Russell and Nurse 1986) and Pyp3 (Elledge 1996) cdc2 protein kinase or p34cdc2 (Colasanti et al. 1991; Feiler and Jacobs 1990; Hirt et al. 1991; John et al. 1989; Lee and Nurse 1987; Nurse and Bissett 198 1; Ormrod and Francis 1993) cdc2a protein kinase (Hemerly et al. 1993) cdc2 T14Y15 kinases such as wee1 or p107wee1 (Elledge 1996; Russell and Nurse 1986; Russell and Nurse 1987a; Russell and Nurse 1987b; Sun et al. 1999) mik1 (Lundgren et al. 1991) and myt1 (Elledge 1996); cdc2 T161 kinases such as Cak and Civ (Elledge 1996); cdc2 T161 phophatases such as Kap1 (Elledge 1996); cdc28 protein kinase or p34cdc28 (Nasmyth 1993; Reed et al. 1985) p4OMO15 (Fesquet et al. 1993; Poon et al. 1993) chk1 kinase (Zeng et al. 1998) cds1 kinase (Zeng et al. 1998) growth-associated H1 kinase (Labbe et al. 1989; Lake and Salzman 1972; Langan 1978; Zeng et al. 1998) MAP kinases described by (Binarova et al. 1998; Bogre et al. 1999; Calderini et al. 1998; Wilson et al. 1999).

Other cell cycle control proteins that are involved in cyclin D-mediated entry of cells into G1 from G0 include pRb (Xie et al. 1996; Huntley et al. 1998) E2F, RIP, MCM7 and potentially the pRb-like proteins p107 and p130.

Additional cell cycle control proteins that are involved in the formation of a pre-replicative complex at one or more origins of replication such as, but not limited to, ORC, CDC6, CDC14, RPA and MCM proteins or in the regulation of formation of this pre-replicative complex, such as, but not limited to, the CDC7, DBF4 and MBF proteins.

As used herein, the terms “cell cycle protein” and “cell cycle control protein” include any one or more of those proteins that are involved in the turnover of any other cell cycle control protein, or in regulating the half-life of said other cell cycle control protein. The term “protein turnover” is to include all biochemcial modifications of a protein leading to the physical or functional removal of said protein. Although not limited to these, examples of such modifications are phosphorylation, ubiquitination and proteolysis. Particularly preferred proteins which are involved in the proteolysis of one or more of any other of the above-mentioned cell cycle control proteins include the yeast-derived and animal-derived proteins, Skp1, Skp2, Rub1, Cdc20, cullins, CDC23, CDC27, CDC16, and plant-derived homologues thereof (Cohen-Fix and Koshland 1997; Hochstrasser 1998; Krek 1998; Lisztwan et al. 1998) and Plesse et al in (Francis et al. 1998)).

For the present purpose, the term “cell cycle control genes” includes any one or more of those genes that are involved in the transcriptional regulation of cell cycle control gene expression such as transcription factors and upstream signal proteins. Additional cell cycle control genes are not excluded.

As used herein, the term “cell cycle control genes” further includes any cell cycle control gene or mutant thereof, which is affected by environmental signals such as for instance stress, nutrients, pathogens or by intrinsic signals such as the animal mitogens or the plant hormones (auxins, cytokinins, ethylene, gibberellic acid, abscisic acid and brassinosteroids).

Regulation by SH2A or SH2A-like proteins of gene transcription and/or cell cycle and/or cellular metabolism as discussed supra can be the consequence of protein-protein interaction or of protein-nucleic acid interaction. Modulating the level and/or activity of potential targets acted upon by SH2A or SH2A-like proteins can thus also contribute to indirect alteration of growth of cells, tissues or organs of an organism. This form of indirect alteration of growth of cells, tissues or organs of an organism in which the level and/or activity of targets of SH2A or SH2A-like proteins are modulated can have the advantage of e.g. avoiding the occurrence of unwanted pleiotropic effects associated with modulation of the level and/or activity of SH2A or SH2A-like proteins.

Methods for identification of potential SH2A or SH2A-like protein targets of proteinaceous nature are well known to the skilled artisan and include yeast two-hybrid screens using SH2A or a SH2A-like protein as a bait and immunoprecipitation using antibodies against SH2A or a SH2A-like protein. Identified proteinaceous targets may include endogenous modulators, e.g. inhibitors or activators of SH2A levels and/or activity or levels and/or activity of SH2A-like proteins. Methods for identification of potential SH2A or SH2A-like protein targets of nucleic acid nature are not as straightforward but still feasible and can e.g. include a gene discovery approach (i.e. identification of genes whose expression is influenced by SH2A or a SH2A-like protein) followed by e.g. gel shift analysis of a set of overlapping promoter fragments derived from said discovered genes. Identified nucleic acid targets may include endogenous genes encoding modulators, e.g. enhancers or inhibitors of expression of genes encoding SH2A or SH2A-like proteins or of activity of SH2A or SH2A-like proteins.

Thus, modulating growth or survival of cells under hypoxic conditions may also be accomplished by modulating the level and/or activity of proteins or nucleic acids which interact with SH2A or SH2A-like proteins. Modulating the level and/or activity of such proteins and/or nucleic acid sequences in cells, tissues or organs of an organism allows modulation of the level and/or activity of SH2A or SH2A-like proteins and/or alteration of the adaption of those cells, tissues or organs of the organism to conditions of hypoxia.

Further analysis of the rice SH2A amino acid sequence using the motif database provided with the OMIGA2.0 software (Oxford Molecular) revealed the presence of multiple putative phosphorylation sites: 5 non-overlapping casein kinase II (CK2) phosphorylation sites, 1 protein kinase C (PKC) phosporylation site and 4 (of which 3 non-overlapping) tyrosine (TYR) phosphorylation sites. The location of these sites is indicated in Table 1. Many of the putative phosphorylation sites in the rice SH2A protein are also conserved in SH2A-like proteins from other sources (see FIG. 2). Thus, biological activity and/or targeting for protein turnover of SH2A or SH2A-like proteins is potentially regulated by regulation of its phosphorylation status. Such regulation can be obtained by changing balances in activities of protein kinases and/or protein phosphatases during different developmental and/or environmental conditions. Targeted exchange of phosphorylatable amino acids (such as serine, threonine and tyrosine) for non-phosporylatable amino acids (such as alanine, alanine and phenylalanine, respectively) might thus yield mutant versions of SH2A or SH2A-like proteins exerting constitutive biological activity or inactivity and/or showing increased or decreased proteolytic turnover rates. Thus, in another aspect of the invention, mutant SH2A or SH2A-like proteins are identified in which key phosphorylatable amino acid(s) are exchanged for non-phosphorylatable amino acid(s). Expression of said modified SH2A or SH2A-like proteins in cells, tissues or organs of an organism results in a modulated level and/or activity of the SH2A or SH2A-like proteins and allows the alteration of the adaptation of those cells, tissues or organs of the organism to conditions of hypoxia. Preferably, adaptation of cells is enhanced to hypoxic conditions.

Table 1: Overview of putative phosphorylation sites occurring in rice SH2A protein. Consensus motifs are: (S,T)-X(2)-(D,E) for casein kinase II (CK2); (S,T)-X-(R,K) for protein kinase C (PKC) and (R,K)-X(2,3)-(D,E)-X(2,3)-Y for tyrosine kinase (TYR).

| Type of | | | % Match with |
|---|---|---|---|
| Phos- | First residue of | Sequence of | consensus |
| phorylation | phosphorylation | phosphorylation | phosphorylation |
| Site | Site | site | motif |
| CK2 | 21 | SEED | 100 |
| PKC | 49 | SWR | 100 |
| TYR | 64 | KRIREARGY | 100 |
| TYR | 65 | RIREARGY | 100 |
| CK2 | 73 | SYVD | 100 |
| CK2 | 94 | SFFE | 100 |
| CK2 | 102 | TDEE | 100 |
| TYR | 107 | RYCLEGSGY | 100 |
| TYR | 145 | RFTLDTDNY | 100 |
| CK2 | 189 | SEGE | 100 |

In accordance with the present invention, gibberellin biosynthesis may be induced in a plant cell, protoplast, explant, plant part or plant organ by modulating the level or activity of SH2A or SH2A homolog. Also in accordance with the present invention, anaerobic response proteins in a plant cell, protoplast, explant, plant part or plant organ may be regulated by modulating the level or activity of SH2 or SH2A homolog.

Modulating the level or activity of SH2A or like protein may comprise transforming a plant cell, protoplast, explant, plant part or plant organ with one or more SH2A-like genes. Similarly, modulating the level of expression or activity of SH2A may comprise transforming a cell, organ or embryo of an animal with one or more SH2A-like genes. Of course, bacteria, yeasts, fungi, and other organisms may be transformed using standard recombinant DNA methods. Since it has now been discerned that SH2A-like proteins are ubiquitous in nature, cells of most if not all organisms will have a native gene coding for an SH2A homolog. Thus, for example, one or more SH2A or like genes may be used in the sense or antisense orientation to transform such organism with the result of increasing or decreasing expression of a native SH2A or like protein.

Nucleotide sequences coding for SH2A-like proteins, and used to transform eg., animal or plant material, may therefore be either heterologous (foreign) to cells of a particular plant or animal, or native to such cells. Resultant transgenic plant and animal cells, or cells of other organisms, may comprise SH2A-like genes which are heterologous to the genome of the particular cells and comprise SH2A-like proteins heterologous to the particular organism. Resultant transgenic plant and animal cells or other organisms may also comprise SH2A-like genes which are native to the genome of the particular organism but which are additional to the genome. In addition, the nucleotide sequence for an SH2A or SH2A-like gene may take the form of the sense or antisense orientation relative to a regulatory region directing expression of said nucleotide sequence. Native or heterologous SH2A-like genes may be added to a cell using standard recombinant DNA methods.

The activity of an SH2A or SH2A-like protein may be modulated by exposing cells to or applying to cells of an organism, a compound which interacts with the SH2A or SH2A-like protein, i.e. an SH2A agonist. The level of an SH2A or SH2A-like protein may be modulated by increasing or decreasing expression of the SH2A or SH2A protein. Increases in expression may be accomplished by e.g., the addition of SH2A coding sequences in the sense orientation. Decreases in expression may be accomplished by e.g., the addition of coding sequences in an antisense orientation, by insertion mutagenesis or by other gene silencing methods. Increases in expression may be further achieved by exposing cells or applying to cells or to whole plants or plant parts, a compound which induces expression of the SH2A or SH2A gene product. Ethylene and ethephon may be used to increase accumulation of mRNA transcripts of an SH2A or SH2A-like gene and thus increase expression of SH2A or SH2A homolog.

The present invention provides an SH2A gene from rice (Oryza sativa) which comprises the nucleotide sequence as set forth in SEQ ID NO:1. In another embodiment of the invention, there is provided an SH2A-like gene from rice designated SH2B comprising the nucleotide sequence as set forth in SEQ ID NO:3. Also contemplated by the present invention are SH2A or SH2A-like genes having an insertion, deletion, or substitution of one or more nucleotides when compared to the sequence set forth in SEQ ID NO:1 and which genes maintain the characteristic property of conferring adaptation to hypoxic conditions.

In yet another embodiment of the invention, there is provided an isolated nucleic acid coding for SH2A having the amino acid sequence as set forth in SEQ ID NO:2. In still another embodiment, there is provided an isolated nucleic acid coding for SH2B having the amino acid sequence as set forth in SEQ ID NO:4.

The present invention also provides isolated nucleic acids coding for SH2A homologs and the corresponding amino acids from such plants as tomato (Lycopersicon esculentum), soybean (Glycine max), and cotton (Gossypium hirsutum). The nucleotide sequence for a first SH2A-like gene from tomato is set forth in SEQ ID NO: 5. The amino acid sequence for the corresponding tomato SH2A homolog is set forth in SEQ ID NO:6. A nucleotide sequence for a second SH2A-like gene from tomato is set forth in SEQ ID NO:7. SEQ ID NO:8 sets forth the corresponding amino acid sequence for a second SH2A-like gene from tomato.

The nucleotide sequence for an SH2A-like gene from soybean is set forth in SEQ ID NO: 9. The amino acid sequence for the corresponding soybean SH2A homolog is set forth in SEQ ID NO:10. The nucleotide sequence for a cotton SH2A-like gene is set forth in SEQ ID NO:11 while the corresponding amino acid sequence is set forth in SEQ ID NO:12.

Also provided are nucleotide sequences for human, mouse and zebrafish SH2A-like genes and the corresponding amino acid sequences. The nucleotide sequence for a human SH2A-like gene is set forth in SEQ ID NO:13. The corresponding amino acid sequence is set forth in SEQ ID NO:14. The nucleotide sequence for a mouse SH2A-like gene is set forth in SEQ ID NO: 15 while the corresponding amino acid sequence is set forth in SEQ ID NO: 16.

The nucleotide sequence for a zebrafish SH2A-like gene is set forth in SEQ ID NO:17. The corresponding amino acid sequence is set forth in SEQ ID NO:18.

An SH2A protein or homolog may be isolated from a tissue source of an organism using well known methodologies. For example, protein extracts may be prepared according to standard procedures, using appropriate extraction buffers as described in Calderini et al. (1998) Journal of Cell Science 111:3091-3100 and the SH2A or SH2A-like protein immunoprecipitated using an antibody. For example, a polyclonal antibody may be produced against a synthetic peptide encoding a portion of SH2A or SH2A-like protein. Thus, a synthetic peptide corresponding to the 3′ region of an SH2A-like gene may be used to generate antibodies to SH2A or SH2A-like protein. For example, a peptide corresponding to nucleotides 496-867 of SH2A may be used to generate antibodies. Peptides corresponding to amino acids 91 to 118 and 133 to 161 of SH2A or SH2A homolog (see FIG. 2) may also be used to generate antibodies. The antibodies may then be used in binding assays with protein extracts from a plant in order to identify an SH2A homolog.

In addition, the present invention relates to antibodies specifically recognizing SH2A or SH2A-like molecule or parts thereof, i.e. specific fragments or epitopes, of such proteins. The antibodies of the invention can be used to identify and isolate SH2A homologs from different organisms. These antibodies can be monoclonal antibodies, polyclonal antibodies or synthetic antibodies as well as fragments of antibodies, such as Fab, Fv or scFv fragments or heavy chain camel antibodies etc. Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988. These antibodies can be used, for example, for the immunoprecipitation and immunolocalization of SH2A and SH2A-like proteins according to the invention as well as for the monitoring of the synthesis of such proteins, for example, in recombinant host cells and organisms. Antibodies or fragments thereof to the aforementioned proteins, peptides or fragments thereof can also be applied in a kit, e.g. for diagnosis of cancer. The alteration of levels of SH2A or SH2A-like proteins in tumours or tumourous tissues could be measured via e.g. a RIA- or ELISA-based detection comprising said antibodies or labelled derivatives thereof provided in the diagnostic kit. In another embodiment of the invention, such a kit for e.g. diagnosis of cancer contains oligonucleotide primers which can be used, e.g. via RT-PCR, for specific and quantitative amplification of the target SH2A or SH2A-like mRNA species. Alteration in the levels of said mRNAs can be indicative for the development of a tumour or of tumourous tissue.

Preferably, the SH2A or SH2A-like protein is isolated from a recombinant organism expressing the gene product of an SH2A or SH2A-like coding sequence of the present invention. SH2A or SH2A-like proteins may be isolated and purified from such recombinant organism using standard methods such as those described in Ausubel et al. (1987) Current Protocols in Molecular Biology, Green Publishing Associates, New York.

Also in accordance with the present invention, there are provided vectors comprising a subject isolated SH2A or SH2A-like nucleotide sequence. The nucleotide sequence may comprise for example, an entire SH2A or SH2A-like gene, i.e, an SH2A genomic sequence comprising coding sequence and associated 5′ and 3′ regulatory regions such as the promoter and termination sequence. Introns may or may not be present in such sequences. In another embodiment, the vectors comprise an SH2A or SH2A-like nucleotide sequence such as a cDNA, operably linked to a promoter which functions in a host cell to effect expression of SH2A or SH2A-like protein. Such a vector may be referred to as a genetic construct. As used herein, “gene” may refer to a genomic sequence, a cDNA sequence or a synthetic DNA sequence coding for SH2A or SH2A homolog in either sense or antisense orientation.

In another aspect of the invention, there is provided a method for producing a host cell which is adapted to growth in low oxygen, i.e., hypoxic conditions. The method involves transforming a host cell with an SH2A or SH2A-like gene under the control of a promoter which functions in the host cell and selecting those transformants with modulated expression levels of the SH2A or SH2A-like gene and which are therefore adapted to grow in low oxygen conditions. The SH2A or like gene may be carried by a vector which replicates in the host cell or which integrates into the genome of the host cell. The method is particularly useful for single cell organisms such as those used in the baking or fermentation industries and in the production of recombinant proteins. Thus, bacteria, fungi, yeasts and animal cells may be made adapted to grow in hypoxic conditions by transformation with a subject SH2A or SH2A-like gene. Examples of bacteria which may be altered by transformation with an SH2A or like gene include but are not limited to E. coli, Bacillus sp., Aquifex sp., and Pseudomona sp. Lactobacilli, Streptomyces sp., Acinetobacter sp., Citrobacter sp. and Clostridium sp. Examples of yeasts which may be altered by transformation with an SH2A or like gene include but are not limited to Saccharomyces, and Schizosaccharomyces, Pichia, and Kluyveromyces and Candida. Examples of fungi which may be made adapted for growth in low oxygen conditions include Aspergillus sp., Penicillium sp. and Trichoderma sp.

An example of animal/mammalian cells which may be altered by transformation with an SH2A or like gene includes but is not limited to hybridoma cells or permanent CHO cells, e.g. producing monoclonal antibodies. Oxygen supply is indeed one of the major problems in the production of useful proteins by cultured animal/mammalian cells (Masuda et al. 2000). Thus, expression of SH2A or SH2A-like genes would enhance survival of said cultured cells. In addition, the promoter of a SH2A or a SH2A-like gene could be used to drive expression of said useful proteins by e.g. animal cells cultured under low oxygen concentrations. Such a system using another hypoxia-response enhancer has been described (Masuda et al. 2000).

An SH2A or SH2A homolog may also be produced in a host cell such as a mammalian, yeast or insect cell by transforming a mammalian, yeast or insect cell with a subject SH2A or SH2A-like gene. Methods of transforming mammalian, yeast or insect cells are well known in the art. In order to produce SH2A or like protein in a mammalian, yeast or insect cell such a cell is transformed with a vector comprising an SH2A gene or SH2A-like gene under the control of a promoter which functions in a mammalian, yeast or insect cell. Such a vector may be referred to as a genetic construct.

In another aspect of the invention, there is provided a method for producing an animal which is adapted to growth in low oxygen, i.e., hypoxic conditions. Methods of producing transgenic animals are well known in the art, the most prevalent method involving the injection of the desired DNA, in this case an SH2A or SH2A-like nucleotide sequence, into the pronucleus of a fertilized embryo. The embryo is transferred to a foster mother where it develops until birth. Tissue specific promoters are widely available and may be chosen for expression of an SH2A or SH2A-like nucleotide sequence. Examples include the sheep major milk whey protein β-lactoglobulin (BLG) gene which is specific for mammary gland expression (Whitelaw et al. 1991 Transgenic Research 1:3-13) and the mouse creatine kinase promoter specific for muscle tissue (Frank et al. 1995 J. Clin. Inves. 96:976-986). For expression in many different cell types, the metallothionein (MT), collagen, and various viral promoters may be used. Transgenic animals exhibit improved growth under conditions of hypoxia.

The methods of the present invention are particularly well suited for use in plant tissue culture, agriculture and horticulture. Thus, the present invention also provides a method of producing a plant which is adapted to growth under conditions of low oxygen, and involves transforming a plant cell, pollen, protoplast, explant, plant part or plant organ with an SH2A or SH2A-like gene, regenerating a transgenic plant therefrom, and selecting for growth under hypoxic conditions. Transgenic plants have improved growth under conditions of low oxygen such as occurs in poorly drained soils and flooded growth conditions.

In still another aspect of the invention, there is provided a method of producing a plant cell or plant protoplast with improved survival in conditions of low oxygen. The method is similar to that involved in improving survival of a plant in low oxygen conditions except that the step of regenerating a transgenic plant is not performed. The plant cells may be stably or transiently transformed with an SH2A or SH2A gene. Such transformed cells are useful for further production of other protein products, including recombinantly produced proteins.

Yet another aspect of the invention provides a method for producing plant somatic embryos with improved survival in conditions of low oxygen. The method is similar to that involved in improving survival of a plant in low oxygen conditions except that transgenic calli are collected and used for induction of somatic embryogenesis. Said method provides advantages comprising increased efficiencies of e.g. batch production of somatic embryos, e.g. from oil palm, and increased survival and germination rates of encapsulated somatic embryos.

Thus, plant cells may be engineered for controlled expression of an SH2A or SH2A-like gene in conditions of low oxygen supply such that the pathways that confer resistance to hypoxic conditions will be induced. Expression of SH2A or SH2A homologs may be targeted to the roots and to other plant parts which are flooding prone to specifically improve resistance to hypoxia in these tissues. The trait which is conferred by an SH2A or SH2A-like gene can be transferred into any crop plant and into any horticulturally important plant as desired.

In addition to the nucleotide sequences provided herein as SEQ ID NOs: 1-18, other nucleotide sequences for SH2A-like genes and corresponding amino acid sequences which are useful in the practice of the present invention are provided on the genetic sequence databases such as EMBL and GenBank. For example, a nucleotide sequence for a second SH2A-like gene in soybean is provided by the accession A1441185 from the Genbank data base. A nucleotide sequence for an SH2A-like gene in cabbage is provided by accession L38235 from the Genbank data base. An SH2A-like gene nucleotide sequence in corn (Zea mays) is provided by accession AI649530 from the Genbank data base. Accession AI054437 from the Genbank data base provides the nucleotide sequence for an Iceplant (Mesembryanthemum crystalllinum) SH2A-like gene. Accession AT000213 from the DDBJ data base provides the nucleotide sequence for an SH2A-like gene in apple (Malus domestica). The nucleotide sequence for an SH2A-like gene in pine (Pinus taeda) is provided on the Genbank database by accession AI813053. Accession AI727947 on the Genbank database provides the nucleotide sequence for a second SH2A-like gene in cotton. Four accessions related to four SH2A-like genes in Arabidopsis thaliana are also available as accessions N38691, N96935, T76549 and AC002505 on the Genbank database and correlate to the ATH1, ATH2, ATH4, and ATH3 genes respectively, as described herein.

In accordance with the present invention, it has been discovered that ATH3 is an essential gene as evidenced by ATH3 knockouts not being viable (Example 16). Since ATH3 is an essential gene, it may be used as a target in herbicide screening procedures.

In one aspect of this invention, interactors of the ATH3 gene product are identified that can be used as plant growth regulator or herbicide. A possible method to identify such interactors of the ATH3 gene product comprises real-time measurement of interactions between said compounds and the ATH3 gene product using the BIACore apparatus (Pharmacia). Preferably said interactors are chemical substances which can find uses as e.g. plant growth regulators or herbicides. As such, the invention also relates to the use of a molecule identified by means of a method as described above as a plant growth regulator or herbicide. According to another embodiment, the invention also relates to a method for production of a plant growth regulator or herbicide composition comprising the steps of the methods described above and formulating the compounds obtained from said steps in a suitable form for the application in agriculture or plant cell or tissue culture.

Accession AI417749 from the Genbank data base provides the nucleotide sequence for a second human SH2A-like gene. A rabbit SH2A-like gene sequence is provided by accession C82769 from the DDBJ database. The nucleotide sequence for an SH2A-like gene from Drosophila is provided by accession AI517276 from the Genbank database. Three different SH2A-like genes in Caenorhabditis elegans are provided by accessions Z68116 from the EMBL database, U80455 from the EMBL database, and U23173 from the EMBL database.

Accession Z48613 from the EMBL database provides the nucleotide sequence for a Saccharomyces cerevisiae SH2A-like gene. Accession AA783142 from the Genbank database provides the nucleotide sequence for an SH2A-like gene from Emericella nidulans. Accession AL033388 from the EMBL database provides the nucleotide sequence for a Schizosaccharomyces pombe SH2A-like gene. Accession Z99111 from the EMBL database provides the nucleotide sequence for a Bacillus subtilis SH2A-like gene. Accession AE000766 from Genbank provides the nucleotide sequence for an SH2A-like gene from Aquifex aeolicus. Accession P28809 from the SWISS-PROT database provides the protein sequence for an SH2A-like gene from Pseudomonas aeroginosa.

SH2A and SH2A-like coding sequences and genomic clones may be obtained by screening a cDNA or genomic library with an appropriate probe. For example, an SH2A genomic clone is obtained by screening a genomic library with the rice SH2A cDNA provided herein or a fragment thereof. An oligonucleotide comprising sequence from the SH2A cDNA may also serve as probe. For example, cDNA fragments covering nucleotides 496-867 of SH2A may be used.

SH2A homologs in plants share between about 70 and about 95% identical amino acids. Oligonucleotide probes based on the nucleotide sequence of one or more plant SH2A-like genes may therefore be designed and synthesized for use in screening cDNA and genomic libraries in order to isolate an SH2A-like gene for use in the present invention. For example, oligonucleotides comprising sequence corresponding to the highly conserved regions of amino acids 99 to 118 and 133 to 161 of the SH2A protein may be used.

Thus, nucleic acid molecules corresponding to coding sequences, promoters or 3′ termination sequences of an SH2A or like gene may also be obtained by using a gene having a sequence as set forth in any of SEQ ID NOs:1-18, including the entire coding sequence of an SH2A or like gene, or portions of an SH2A-like coding sequence (including fragments and oligonucleotides) as a probe and hybridizing with a nucleic acid molecule(s) from a particular organism. By “hybridizing”, it is meant that such nucleic acid molecules hybridize under conventional hybridization conditions, such as described by, e.g., Sambrook et al(1989) Molecular Cloning, A Laboratory Manual, 2^{nd} Edition, Cold Spring Harbor Press, Cold Spring Harbor, N.Y.

Nucleic acid molecules hybridizing to Oryza sativa SH2A cDNA, (SEQ ID NO:1), or to any of the nucleotide sequences or parts thereof set forth in SEQ ID NOs:1-18, can be isolated, e.g., from cDNA or genomic libraries by techniques well known in the art. Methods considered useful in obtaining genomic DNA sequences corresponding to SH2A and SH2A-like gene of the present invention by screening a cDNA or genomic library, are provided in Sambrook et al. (1989), Molecular Cloning. A Laboratory Manual, Cold Spring Harbor, N.Y., for example, or any of the myriad of laboratory manuals on recombinant DNA technology that are widely available.

A subject SH2A or like gene can be derived from restriction endonuclease digestion of isolated SH2A or SH2A-like genomic clones. Thus, for example, the known nucleotide or amino acid sequence of the rice SH2A gene (SEQ ID NO: 1) is aligned to the nucleic acid or deduced amino acid sequence of an isolated putative SH2A-like genomic clone and the 5′ regulatory sequence (i.e., sequence upstream from the translational start codon of the coding region), coding sequence, and 3′ regulatory sequence (i.e., sequence downstream from the translational stop codon of the coding region) of the isolated SH2A or SH2A-like genomic clone located.

An SH2A or like gene may be generated from genomic clones having excess 5′ flanking sequence, excess coding sequence and/or excess 3′ flanking sequence by e.g., in vitro mutagenesis. In vitro mutagenesis is helpful for introducing convenient restriction sites. There are various commercially available kits particularly suited for this application such as the T7-Gen in vitro mutagenesis Kit (USB, Cleveland, Ohio) and the QuikChange Site Directed Mutagenesis Kit (Stratagene, San Diego, Calif.). Alternatively, PCR primers can be defined to allow direct amplification of an SH2A or like gene, including the promoter, coding sequence and 3′ termination sequence.

An SH2A or like gene for use in the present invention is not necessarily isolated from a gene library but may be generated in any manner, including for example, chemical synthesis, DNA replication, transcription, and reverse transcription. Thus, as used herein, SH2A and SH2A-like genes encompass sequences made up of both ribonucleotides and deoxyribonucleotides.

The general techniques used in the subject invention, especially in preparing and probing a cDNA or genomic library, sequencing isolated clones, performing deletion analysis, constructing expression vectors, transforming cells and growing cells and the like are known in the art and laboratory manuals describing such techniques are widely available. See eg. Sambrook et al. 2^{nd} Ed., (1989) Molecular Cloning: A Laboratory Manual, Cold Spring Harbor, N.Y.

The strong conservation of the SH2A and SH2A-like gene product in eukaryotes in general indicates an evolutionary conserved function. Thus, any SH2A or SH2A-like gene may be used in the methods of the present invention. Preferably, cells are transformed with the phylogenetically closest SH2A-like gene available. For example, plant cells, protoplasts, explants, plant parts, and organs are preferably transformed with a plant SH2A or like gene such as the SH2A gene from Oryza sativa (SEQ ID NO:1), the ATH1 (accession N38691), ATH2 (accession N96935), ATH3 (accession AC002505), or ATH4 (accession T76549) genes from Arabidopsis thaliana or other SH2A-like gene such as tomato 1 (SEQ ID NO:5), tomato 2, (SEQ ID NO:7) soybean 1 (SEQ ID NO:9), soybean 2 (accession A1441185), cotton 1 (SEQ ID NO:11), cotton 2 (accession A1727947), or other SH2A-like and/or corresponding genomic sequences isolated from plants.

In accordance with the present invention, the SH2A or SH2A-like coding sequence may correspond to a cDNA or genomic sequence. When a genomic SH2A or SH2A-like gene is used, the gene may be altered, if desired, to remove or add one or more introns. In addition, signal sequences may be removed and/or added to the genomic sequence. Signal sequences and/or intron(s) may also be added to an SH2A or SH2A-like cDNA. Nucleotide sequences for an SH2A or like gene, which may include one or more introns, may be operably linked to a promoter from the same SH2A or like gene or to a promoter from another SH2A gene. Alternatively, the SH2A or like nucleotide sequences may be operably linked to a promoter which functions in plant cells but which is unrelated to an SH2A or like gene. An example of another promoter responsive to hypoxic conditions is e.g. a synthetic promoter comprising e.g. a repeat of 6 AREs (ARE=anaerobic responsive element) of the maize Adh1 gene (Olive et al. 1990).

Examples of promoters which function constitutively in plant cells include the cauliflower mosaic virus (CaMV) 35S promoter, nopaline synthase (nos) promoter, Aslfalfa Mosaic Virus (AMV) promoter, and enhanced mannopine synthase promoter (MAC) promoter. These promoters are well characterized and widely available. Examples of inducible promoters which may be used to control expression of an SH2 gene include heat shock promoters, a nitrate-inducible promoter derived from the spinach nitrate reductase gene (Back et al. 1991 Plant Mol. Biol. 1 7:9), hormone inducible sequences (e.g., Yamagushi-Shinzaki, E. et al. 1990 Plant Mol. Biol. 15:905, Kares et al (1990) Plant Mol. Biol 15:225) and light inducible promoters such as the small subunit of RuBP carboxylase and LHCP gene families. Other examples of inducible promoters include tetracycline-inducible promoters (Gatz et al. 1992 Plant J. 2:397), glucocorticoid-inducible promoters (WO 9938988) and promoters induced by chemicals as described in e.g. EP0332104 and WO 9008826.

Tissue specific and developmentally regulated promoters may also be used to control and regulate expression of an SH2A or like gene. Examples include pollen-specific (Albani et al. 1991 Plant Mol. Biol. 16:501; Twell, et al., 1991 Genes Dev. 5:496; Hamilton D. et al., 1992 Plant Mol. Biol. 18:21 1), flower-specific (van der Meer et al. 1990 Plant Mol. Biol. 15:95, phloem-specific (DeWitt, N. D., et al. 1991 J. Cell Biochem. Suppl. 15A:69, Yang, N. -S. Et al. 1990 Proc. Natl. Acad. Sci. U.S.A., 87:4144), root specific (Depater, B. S. et al., 1992 Plant Mol. Biol. 18:161; Vanderzaal, e., et al. 1991 Plant Mol. Biol. 16:983; Oppenheimer, D. G., et al., 1988 Gene 63:87) and seed-specific promoters (Bustos, M. M., et al., 1991 EMBO J. 10: 1469; Stayton,M., et al., 1991 Aust. J. Plant Physiol. 18:507) including the kappa hydroxylase promoter as described in WO 9903983. Especially preferred promoters include root-specific promoters such that expression of SH2A or SH2A homolog will be targeted to the roots of a plant. Targeting SH2A or like gene expression to the roots is especially useful in increasing submergence tolerance in a less adapted plant species. Such root-specific promoters include promoters of the maize root preferential cationic peroxidase gene (WO 9856921) and the promoter of a sugarbeet storage root-specific gene whose transcription activity is not altered by switching from aerobic to anaerobic conditions (WO 9732027). Meristem-specific promoters, which are well known in the art (e.g. as described in U.S. Pat. No. 5,898,096), may also be used in order to target SH2A or like gene mRNA transcripts in either the sense or antisense orientation to the meristematic regions of a plant.

The present invention also contemplates the corresponding 5′ and 3′ regulatory regions including the promoter and terminator regions of SH2A and SH2A-like genes. Thus, for example, the promoter for SH2A is useful for promoting expression of SH2A or like gene as well as for promoting expression of other heterologous sequences, i.e., coding sequences unrelated to an SH2A or SH2A-like gene. The 5′ regulatory region is induced under conditions of low oxygen supply and is thus useful for promoting expression of a number of different coding sequences under such conditions. In addition, the SH2A or SH2A-like gene promoter may be further induced by exposure to ethylene or its precursor, ethephon.

To provide regulated expression of an SH2A or SH2A-like gene, plants are transformed with a nucleic acid sequence comprising a promoter which functions in plants, which as described above, may include an SH2A or SH2A-like gene promoter or other promoter, operably linked to a cDNA or genomic SH2A or SH2A-like sequence. Usually, the nucleic acid sequence is placed within a vector and may be referred to as a genetic construct. Preferably, the SH2A or SH2A-like gene also comprises a 3′ regulatory sequence. When the SH2A or SH2A-like coding sequence is operably linked to a promoter other than that naturally occurring 5′ upstream from the coding sequence such a nucleotide sequence may be referred to as a chimeric gene or chimeric gene construct. Similarly, when an SH2A or like gene 5′ or 3′ regulatory region is operably linked to coding sequence other than that of the corresponding SH2A or like gene, or to a completely unrelated gene, such a nucleotide sequence is also referred to as a chimeric gene or chimeric gene construct.

Methods of gene transfer in plants are well known in the art (Gelvin 1998 Current Opin Biotech 9:227). The SH2A or like genes including chimeric genes may be introduced into plants by leaf disk transformation-regeneration procedure as described by Horsch et al. (1985) Science, 227; 1229-1231. Other methods of transformation such as protoplast culture (Horsch et al. (1984) Science, 223; 496; DeBlock et al. (1984) EMBO J., 2; 2143; Barton et al. (1983) Cell, 32; 1033) and root transformation (Valvekens et al. 1988 Proc Natl Acad Sci USA 85:5536) can also be used and are within the scope of this invention. In a preferred embodiment, plants are transformed with Agrobacterium-derived vectors such as those described in Klett et al. (1987) Annu. Rev. Plant Physiol., 38; 467. Such transformation vectors include binary-, super-binary, cointegrate- and Ri-derived vectors and T-DNA carrying vectors used in agrolistic transformation. Other well-known methods are available to insert the chimeric genes of the present invention into plant cells. Such alternative methods include biolistic approaches (Klein et al. (1987) Nature, 327; 70), electroporation, microinjection, chemically-induced DNA uptake, and use of viruses or pollen as vectors.

When necessary for the transformation method, an SH2A or like gene or a subject chimeric gene may be inserted into a plant transformation vector, e.g. the binary vector described by Bevan (1984) Nucleic Acids Res., 12; 8711-8721. Plant transformation vectors can be derived by modifying the natural gene transfer system of Agrobacterium tumefaciens. The natural system comprises large Ti (tumor-inducing)-plasmids containing a large segment, known as T-DNA, which is transferred to transformed plants. Another segment of the Ti plasmid, the vir region, is responsible for T-DNA transfer. The T-DNA region is bordered by terminal repeats. In the modified binary vectors, the tumor inducing genes have been deleted and the functions of the vir region are utilized to transfer foreign DNA bordered by the T-DNA border sequences. The T-region also contains a selectable marker for antibiotic resistance, and a multiple cloning site for inserting sequences for transfer. Such engineered strains are known as “disarmed” A. tumefaciens strains, and allow the efficient transfer of sequences bordered by the T-region into the nuclear genome of plants.

Surface-sterilized leaf disks and other susceptible tissues are inoculated with the “disarmed” foreign DNA-containing A. tumefaciens, cultured for a number of days, and then transferred to antibiotic-containing medium. Transformed shoots are then selected after rooting in medium containing the appropriate antibiotic, and transferred to soil. Transgenic plants are pollinated and seeds from these plants are collected and grown on antibiotic medium.

Expression of an SH2A gene, an SH2A-like gene or subject chimeric gene construct in developing seeds, young seedlings and mature plants can be monitored by immunological, histochemical, mRNA expression or activity assays. For assaying expression of an SH2A-like gene, northern analysis may be performed as described in Example 4. When a chimeric gene comprises an SH2A or SH2A-like promoter operably linked to a gene other than an SH2A or SH2A-like gene, the choice of an assay for expression of the chimeric gene depends upon the nature of the heterologous coding region. For example, Northern analysis can be used to assess transcription if appropriate nucleotide probes are available. If antibodies to the polypeptide encoded by the heterologous gene are available, Western, RIA or ELISA analysis and immunohistochemical localization can be used to assess the production and localization of the polypeptide.

Another aspect of the present invention provides transgenic plants, progeny or essentially derived varieties of these plants containing the SH2A, SH2A-like genes and chimeric gene constructs of the invention. Both monocotyledonous and dicotyledonous plants are contemplated. As used herein, an “essentially derived variety” is held to exist where (a) it is predominantly derived from the initial variety, (b) it is distinct from the initial variety and (c) it conforms essentially to the initial variety in the expression of the introduced transgene.

Plant cells are transformed with an SH2 or SH2A-like gene or chimeric gene construct by any of the plant transformation methods described above. The transformed plant cell, usually in the form of a callus culture, leaf disk, explant or whole plant (via the vacuum infiltration method of Bechtold et al. (1993) C.R. Acad. Sci. Paris, 316; 1194-1199) is regenerated into a complete transgenic plant by methods well-known to one of ordinary skill in the art (e.g., Horsh et al., 1985). Since progeny and essentially derived varieties of transformed plants inherit the chimeric genes, pollen, ovum, seeds, tubers or cuttings from transformed plants or essentially derived varieties thereof may be used to maintain the transgenic trait in the transformed line or variety essentially derived thereof.

The present invention also encompasses flowers from a transgenic plant which expresses an SH2A or SH2A-like protein or which comprises an SH2A or SH2A-like chimeric gene construct.

The following examples further illustrate the invention and are not intended in any way to limit the invention.

### EXAMPLE 1

### Plant Material and Incubation Conditions

Seeds of deepwater rice (Oryza sativa L., Pin Gaew 56) were obtained from the International Rice Research Institute (Los Baños, The Philippines). Plants were grown for 12 to 14 weeks as described (Sauter, 1997). All experiments were carried out under continuous light (200 μE/m⁻² s⁻¹) at 25° C. in a growth chamber. For submergence treatment, whole plants were placed in a 600 liter plastic barrel filled with tap water as described (Lorbiecke and Sauter, 1999). Control plants were kept in the same growth chamber. Analysis of hormone and inhibitor effects was performed using excised stem segments containing the upper highest growth responsive internode. (Raskin and Kende, 1984). Stem segments were incubated in 150 ml beakers with 25 ml aqueous solutions containing the indicated concentrations of gibberellin A₃ (GA₃) or the hormone precursor ethephon (2-chlorethanephosphoric acid, E) or water alone as a control. Control segments were incubated for 0.5 or 3 h in water. For ethephon treatment stem segments were preincubated for 0.5 h in water before adding ethephon for 2.5 h.

To guarantee high humidity, the beakers were placed in plastic cylinders. Ethylene action was inhibited using 2,5-norbornadiene (bicyclo[2.2.1]hepta-2,5-diene, NBD) at a concentration of 50 μl/l in the gas phase as described (Lorbiecke and Sauter, 1999).

To inhibit protein synthesis, stem sections were incubated with aqueous solutions of cycloheximide for 3 hours at the concentrations indicated. When using ethephon together with cycloheximide, stem sections were first preincubated for 30 min in cycloheximide solution alone to ensure inhibition of protein synthesis before adding ethephon to a concentration of 150 μM for 2.5 h.

### EXAMPLE 2

### Molecular Cloning and Sequence Analysis of a Submergence-induced Early Response Gene in Rice

A submergence-induced early response gene (SH2) was isolated from rice using a PCR-based subtractive hybridization aimed at identifying submergence-induced genes in adventitious roots of partially submerged deepwater rice plants. PCR-based subtractive hybridization was performed according to the method described by Buchanan-Wollaston and Ainsworth (1997) using cDNAs synthesized from mRNA of adventitious roots of the third node from unsubmerged deepwater rice plants as driver population and from plants partially submerged for 2 h as target population. To obtain full-length cDNAs, screening of a ambda-ZAPII-cDNA library of deepwater rice (Sauter, 1995) was performed according to the “DIG System User's Guide” (Boehringer, Mannheim, Germany) using a digoxigenin-labeled 373 bp cDNA-fragment identified in the PCR-based subtractive hybridization. Six strongly hybridizing plaques were recovered from a total of 2×10⁵ recombinant phages. The clone containing the longest insert was sequenced from both sides by the dideoxynucleotide chain termination method of Sanger et al. (1977) with the ABI PRISM Dye Terminator Cycle Sequencing Kit (Applied Biosystems, Weiterstadt, Germany). The longest clone, designated SH2A (gb AF050200), contained an insert of 872 bp. The nucleotide sequence of SH2A is set forth in SEQ ID NO:1. The sequence between nucleotides 496-867 was identical to the cDNA fragment used for library screening. SH2A encodes an open reading frame of 597 bp. The predicted polypeptide is 199 aa long with a predicted molecular weight of 23.6 kDa. The amino acid sequence corresponding to SH2A is set forth in SEQ ID NO:2. An in-frame stop codon in the 5′-untranslated region at nucleotides 30 to 33 indicated that SH2A comprised the complete coding region of the putative protein.

### EXAMPLE 3

### Computational Analysis

The rice EST-clones S2993 and S12166 were obtained from the Rice Genome Research Program at the National Institute of Agrobiological Resources (Tsukuba, Japan) and sequenced from both sides by the dideoxynucleotide chain termination method of Sanger et al. (1977) with the ABI PRISM Dye Terminator Cycle Sequencing Kit (Applied Biosystems, Wieterstadt, Germany). DNA sequence data were analysed and virtually translated using the DNasis V 5.11 program (Hitachi Software Engineering Co., Ltd. 1984, 1991). Homologs were searched in the actual releases of the Swiss-Prot, TrEMBL (Bairoch and Apweiler, 1998), EMBL (Stoesser et al., 1998), GenBank (Benson et al., 1998), DDBJ (Tateno et al., 1998) and PIR (Barker et al., 1998) databases with the BLAST 2.0.3 program (Altschul et al., 1997). Identified ESTs representing the same gene were cloned in silico to obtain complete sequence information of the putative open reading frame. Alignments of the sequences were calculated with the CLUSTAL X 1.64b program (Thomson et al., 1997) and manually edited using GeneDoc 2.1 (Nicholas and Nicholas, 1997). Phylogenetic analysis was done using CLUSTAL X 1.64b and Treeview 1.31 (Page, 1996). The putative secondary structure of SH2A came from the consensus of calculations using six different prediction programs (Ito et al., 1997; White et al., 1994; Rost and Sander, 1993,1994; Gibrat et al., 1987; Chou and Fasman, 1978; Kneller et al., 1990). Only regions where four out of six prediction programs gave similar results were taken into consideration.

Database homology searches led to the identification of three rice ESTs identical to SH2A and four EST clones representing one close sequence homolog of SH2A. Two of the ESTs representing the SH2A homolog, S2993 and S12166, were obtained from the Rice Genome Research Program and completely sequenced. S2993, designated SH2B (gb AF068332), contained an insert of 980 bp and an open reading frame coding for 198 amino acids. An in-frame stop codon was located in the 5′-untranslated region. The S12166 sequence was identical to SH2B with the exception of a 64 nucleotide extension preceding the poly A⁺-tail suggesting alternative polyadenylation of SH2B.

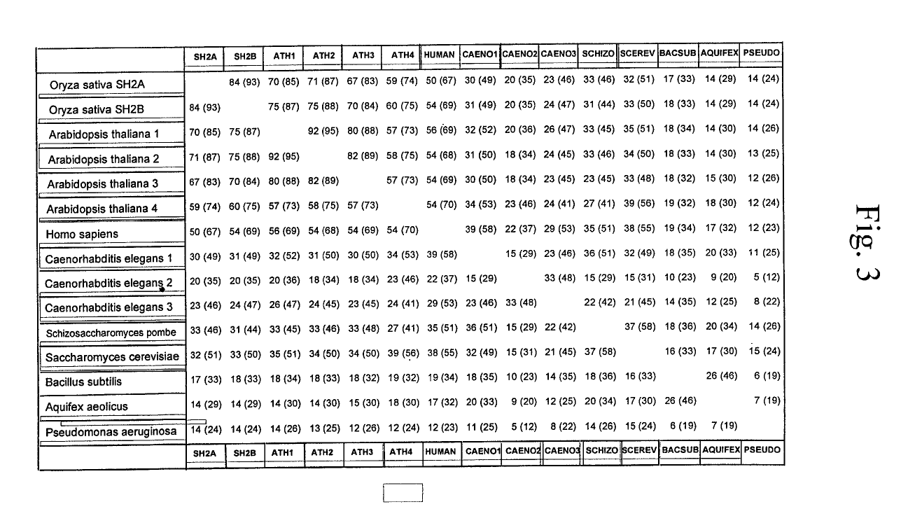

The nucleotide sequence homology between the coding regions of SH2A and SH2B was 84%. No significant homology was observed in the 5′- and 3 ′-untranslated regions of the two clones. Further analysis revealed that SH2A and SH2B are members of a novel class of highly conserved proteins. A computer search of nucleotide and protein data bases indicated that the deduced open reading frame of SH2A exhibited significant homology to putative proteins and putative open reading frames of a number of ESTs coresponding to hypothetical proteins of other plants, animals and fungi (FIG. 2). Analysis of ESTs and genomic sequences led to the identification of four transcribed SH2 homologs in Arabidopsis thaliana. In addition, ESTs of close SH2 homologs from other dicotyledonous plants were identified. Comparison of the putative full-length sequences of SH2 homologs from plants revealed an amino acid identity of 57% to 92% (FIG. 3). The deduced amino acid sequence of a human homolog was 50% identical and 67% similar to SH2A. Amino acid identity between putative proteins of Saccharomyces cerevisiae and Schizosaccharomyces pombe and SH2A were 32% and 33%, respectively. Most of the eukaryotic sequences contained a putative nuclear localization signal (KRXR at aa position 64 to 67 of SH2A). Some sequences, including SH2A and SH2B contained an R(X)₂L(X)₂,₃N destruction box motif (Glotzer et al., 1991).

A weaker homology was observed when SH2A was compared to three bacterial proteins from Bacillus subtilis, Aquifex aeolicus and Pseudomonas aeruginosa (FIG. 2). The Pseudomonas aeruginosa protein encoded by the mmsR gene is a positive regulator for the methylmalonate semialdehyde dehydrogenase operon (Steele et al., 1992). mmsR has been identified as a member of the XylS/AraC family of positive bacterial transcriptional regulators. Most family members contain a conserved DNA binding domain usually located at the C-terminus that is connected to a nonconserved region by a linker (Gallegos et al., 1997). The homologous region between SH2A and mmsR is located in the nonconserved domain of mmsR. In addition, a hydropathy analysis (Kyte and Doolittle, 1982) of the region suggested a structural similarity (FIG. 4).

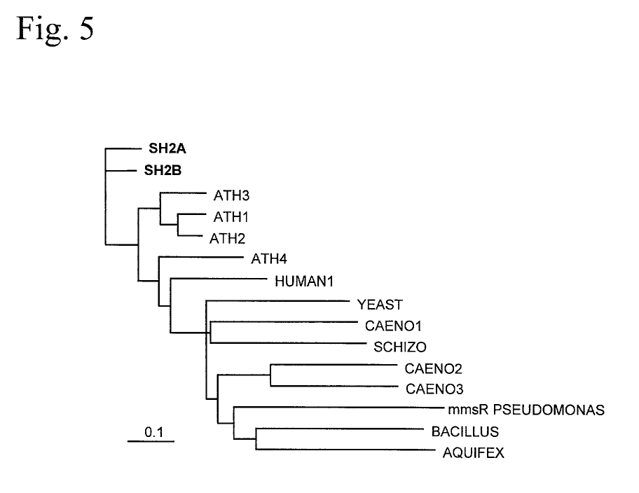

Comparison of the aligned SH2-related sequences determined the highest conserved regions between aa 91 to 118 and 133 to 161 of the SH2A protein (FIG. 2). The region between aa 133 to 161 was characterized by a high content of hydrophobic residues and a putative_-helix followed by two putative_-sheets. A proline residue (P 139 of SH2A) was conserved in all sequences. Whereas the N-terminal regions of SH2 homologs from plants, higher animals and human were very similar, this region was less conserved in the sequences of Caenorhabditis elegans, fungi and bacteria. This was reflected in the phylogenetic relationship calculated from the available full-length sequences (FIG. 5).

### EXAMPLE 4

### Submergence Responsive Expression of SH2A and SH2B

To determine submergence-dependent gene expression, mRNA abundance of SH2A and SH2B was analysed by RNA gel blot hybridization. RNA was isolated from different zones of the youngest internode and from adventitious roots of the third node from airgrown or partially submerged deepwater rice plants. Thus, tissue sections of the highest internode, 0-5 mm above the second node and containing the intercalary meristem; 5-15 mm above the second node containing the elongation zone; and 15-30 mm above the second node and containing the differentiation zone were used (Lorbiecke and Sauter, 1997). Adventitious roots of the third node were isolated as described (Lorbiecke and Sauter, 1999). The tissues were immediately frozen in liquid nitrogen and stored at −70° C. until RNA extraction. Total RNA was isolated with the TRIzol reagent (Gibco BRL) and precipitated with 4 M LiCl as described (Puissant and Houdebine, 1990, Lorbiecke and Sauter, 1999). RNA separation and Northern blot hybridization were carried out as described previously (Lorbiecke and Sauter, 1998). For RNA blot analysis, 20 μg of total RNA was separated by electrophoresis on a 1% (w/v) agarose gel containing 6% formaldehyde. The RNA was blotted to a nylon membrane (Hybond N+; Amersham, Braunschweig, Germany) according to Lorbiecke and Sauter (1998). For hybridization, gene-specific cDNA-fragments covering the 3′-regions of SH2A (nt 496-867) and SH2B (nt 513-980) were random prime labelled with 32P using the ‘Rediprime Labelling Kit’ (Amersham, Braunschweig, Germany). Hybridization was carried out overnight at 68° C. in 1% SDS, 1 M NaCl, 10% (w/v) dextran sulfate and heat-denatured salmon sperm DNA. The blots were washed once in 1× SSC (0.15 M NaCl, 15 mM Na-citrate, pH 7.0) at 68° C. and once in 1× SSC, 1% (w:v) SDS at 68° C. for 10 min each (Sauter, 1997).

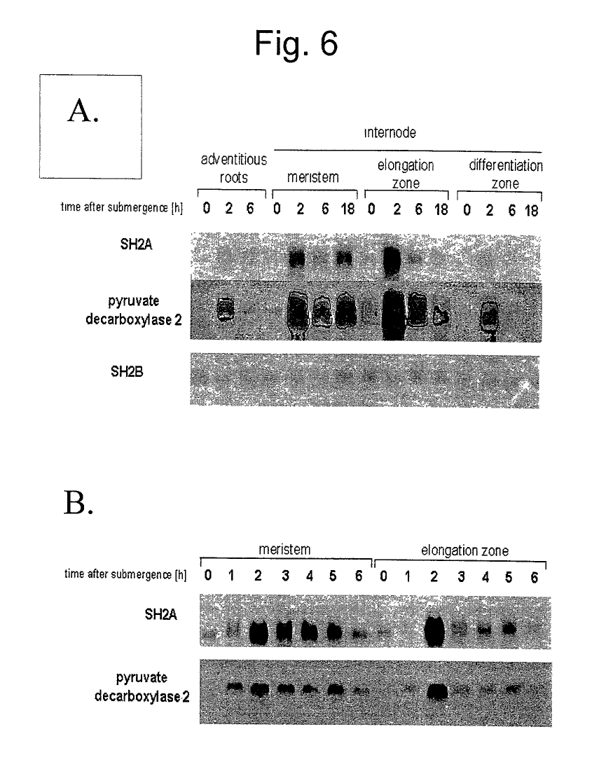

SH2A expression was transiently induced in the tissues analysed. Strongest induction was observed in the intercalary meristem (IM) and in the elongation zone (EZ) of the youngest internode (FIG. 6A). Fewer transcripts accumulated in the differentiation zone and in adventitious roots. mRNA abundance of SH2A increased between 0 h and 1 h in the intercalary meristem and between 1 h and 2 h in the elongation zone, respectively. Maximal mRNA abundance occurred 2 h after submergence in both tissues (FIG. 6B). Between 2 h and 6 h after submergence, SH2A mRNA transcripts dropped to control levels. By contrast, Northern analysis using a gene-specific probe of SH2B revealed no significant changes in mRNA abundance in any of the tissues at the time points analysed (FIG. 6A), indicating constitutive expression of this gene.

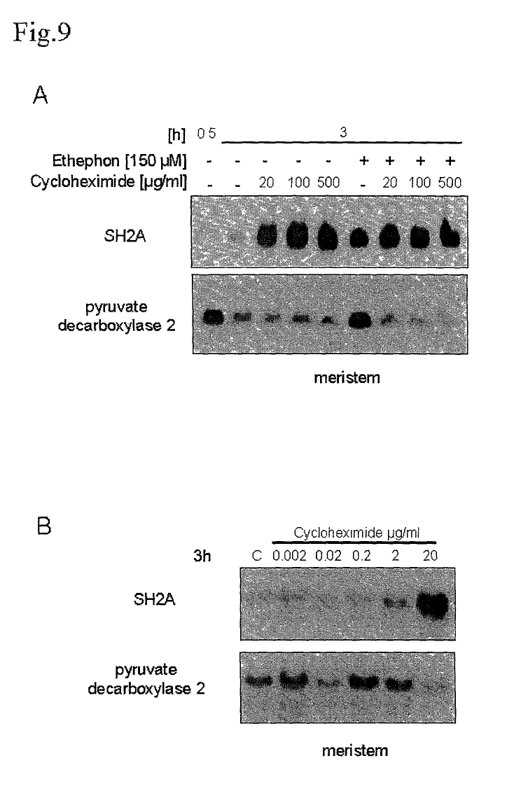

SH2A expression and expression of the key anaerobic response protein pyruvate decarboxylase 2 is closely similar in submergence induced rice plants (FIGS. 6a and 6b). Pyruvate decarboxylase 2 gene expression in the internode is regulated by ethylene as is SH2A gene expression. Unlike SH2A however, pyruvate decarboxylase 2 is not an early response gene. Pyruvate decarboxylase 2 expression depends on protein synthesis (Example 6, FIG. 9). The foregoing results indicate that the SH2A gene product is involved in regulating anaerobic response proteins including pyruvate decarboxylase and is therefore elemental to control of submergence or water logging tolerance.

### EXAMPLE 5

### Genomic Organization of SH2A and SH2B

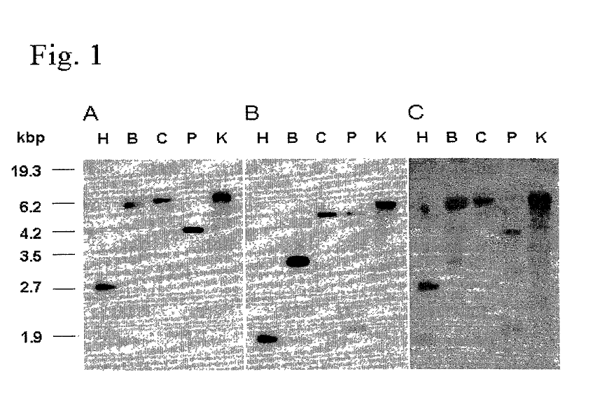

Genomic DNA was isolated from the youngest leaf of 11 week old deepwater rice plants as described (Dellaporta et al., 1983), digested for 5 to 6 h with BamHI, ClaI, HindIII, KpnI or PstI and separated by electrophoresis on a 1% (w/v) agarose gel. The DNA was capillary blotted to a nylon membrane (Hybond N+; Amersham, Braunschweig, Germany) and hybridized with gene-specific probes under high stringency conditions as described for Northern analysis (Example 4). To detect SH2-related sequences, hybridization was performed under less stringent conditions using the SH2A cDNA as a probe. Southern analysis under low stringency conditions was carried out as described for Northern analysis (Example 4) except that hybridization and washing steps were performed at 55° C. instead of 68° C. To detect SH2-related sequences hybridization was performed under less stringent conditions using the SH2A-cDNA as a probe. Southern blot analysis of rice genomic DNA with a gene-specific probe of SH2A detected a single band with five different enzymes when hybridized and washed at high stringency (FIG. 1a). A gene-specific probe of SH2B detected a single band with four different enzymes and two bands with PstI digested DNA when hybridized under stringent conditions (FIG. 1b). No PstI restriction site of was present in the cDNA sequence indicating the existence of at least one intron in the SH2B gene. These results indicate that SH2A and SH2B represent single copy genes in the rice genome. In a Southern analysis using the complete cDNA of SH2A with the same blot under low stringency conditions, nearly all visible bands were attributed to signals detected with SH2A or SH2B gene-specific probes, indicating that there are no additional close SH2 homologs in rice (FIG. 1).

### EXAMPLE 6

### Induction of SH2A Gene Expression

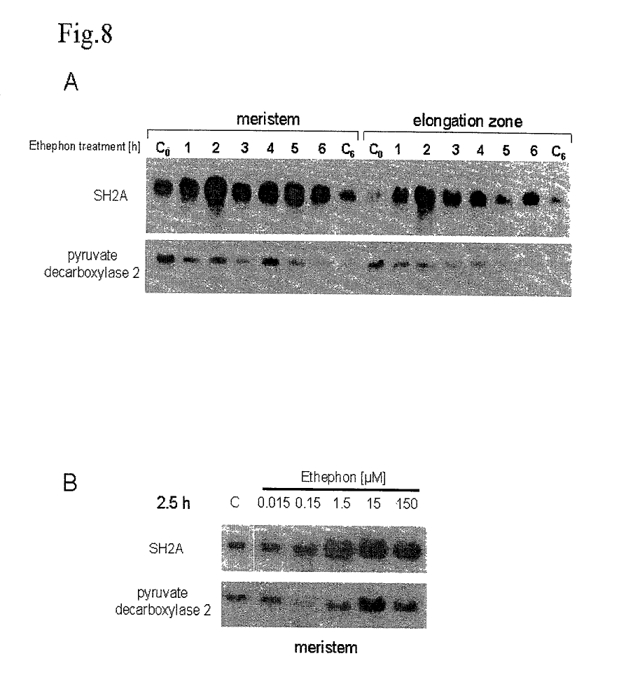

To analyse the hormone-dependent expression of the SH2A gene, isolated stem segments containing the youngest internode were treated with different hormone or inhibitor solutions for 2.5 hours. Total RNAs were isolated from the regions containing the intercalary meristem and the elongation zone and were analysed for SH2A transcript abundance (FIG. 8a). SH2A expression was slightly induced in controls which was likely due to excision of the tissue from the plant.

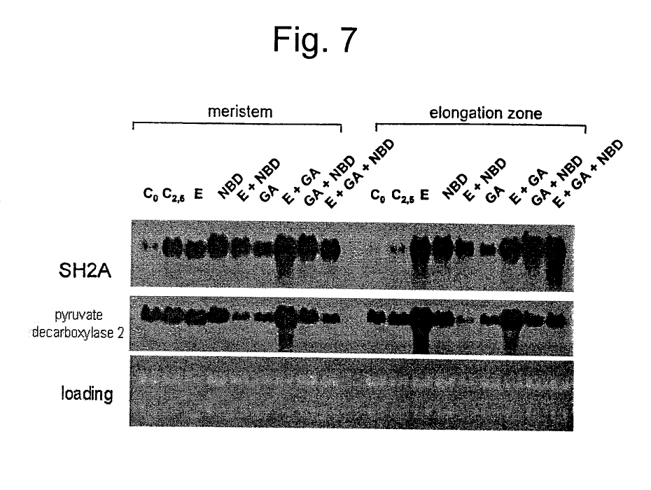

Treatment of stem segments with 150 μM ethephon (2-chloroethanephosphoric acid) led to a strong increase in SH2A transcript levels. When using ethephon in combination with an inhibitor of ethylene action, norbornadiene (applied at a concentration of 50 μl/l in the gas phase), the SH2A transcript levels were reduced to control levels (FIG. 7). These results indicate that the SH2A gene is regulated by ethylene. Incubation with 50 μM GA₃ did not result in increased SH2A transcript levels after 2.5 h when compared with controls. A combination of GA₃ and ethephon or GA and norbornadiene gave similar results as without GA₃ (FIG. 7).

To determine the pattern of ethephon-dependent SH2A expression, total RNA was isolated at 1 hour intervals from the meristem and from the elongation zone of stem segments incubated with 150 μM ethephon. Northern blot analysis showed an increase of SH2A mRNA between 0 hours and 2 hours in both tissues (FIG. 8A). Maximal mRNA abundance was detected after incubation for 2 hours. Between 2 and 6 hours, mRNA levels declined to control levels. Comparison between isolated stem segments treated with ethephon and partially submerged intact plants (FIG. 6B) revealed a similar pattern of transient SH2A expression in the intercalary meristem and in the elongation zone, respectively.

To determine the dose-dependent response of SH2A gene expression, isolated stem segments were incubated with different ethephon concentrations for 2.5 hours. Ethephon concentrations used were 0.015, 0.15, 1.5, 15, and 150 μM. Northern blot analysis of the RNA isolated from the intercalary meristem showed that SH2A transcript accumulation was induced with as little as 1.5 uM ethephon (FIG. 8B). Induction of SH2A gene expression occurred in the presence of cycloheximide (CHX), a known inhibitor of protein synthesis. This observation indicates that the SH2A gene is induced without prior de novo protein synthesis. Treatment of stem segments with CHX alone resulted in accumulation of SH2A transcripts (FIG. 9A). This phenomenon is frequently observed with early response genes and has been termed superinduction. Dose response tests revealed that cycloheximide concentrations of 20 μg/ml and higher were required for gene induction. Such concentrations have been reported to effectively block protein synthesis possibly indicating that SH2A gene expression is normally suppressed by a labile protein (FIG. 9B).

The foregoing findings of: (i) time course of SH2A gene expression (FIGS. 6A and 6B); (ii) regulation of transcription by ethylene but not gibberellin (FIG. 7); (iii) the location of gene induction in young tissues; and (iv) identification of SH2A as an early response regulator, indicate that the SH2A protein is a signal component that regulates gibberellin homeostasis, resulting in altered levels of gibberellin in the rice stem. Through its effect on gibberellin levels, SH2A alters the growth response in rice.

### EXAMPLE 7

### Evidence for Hormone-regulated Gene Expression of Arabidopsis SH2A-like Genes

Sequence analysis of the promoter regions of three SH2A-like genes from Arabidopsis thaliana, ATH1 (accession Z97336), ATH2 (accession Z97336) from the EMBL database, and ATH3 (accession AC002505) from the GenBank database, revealed several cis-acting elements for which participation in signal-dependent gene regulation has been shown in other genes (Dolferus et al. 1994, Gubler and Jacobsen 1992, Gubler et al. 1995, Manjunath and Sachs 1997, Ohme-Takagi and Shinshi 1995, Olive et al. 1990). The elements identified in Arabidopsis involve response to anaerobiosis, ethylene, GA or ABA. A schematic drawing of putative cis-elements in the promoter regions of three SH2A-like genes from Arabidopsis thaliana is depicted in FIG. 10.

### EXAMPLE 8

### Analysis of SH2A-like Gene Expression in Yeast (Saccharomyces cerevisiae) and Animal Cells Under Conditions of Hypoxia

Yeast cells are grown at 3 0C in minimal medium under normoxic conditions (21% O₂, 5% CO₂, 75% N₂) and under different oxygen concentrations in an oxygen-regulated incubator.

Animal cells, e.g. Chinese hamster ovary (CHO) cells, are maintained in Minimum Essential Alpha Medium supplemented with ribonucleotides, deoxyribonucleotides (Gibco, N.Y.) and 10% fetal calf serum under normoxic conditions in a humidified incubator at 37° C. An oxygen-regulated incubator generating different oxygen concentrations with balanced N₂ is used to culture cells under low oxygen (10%, 5% and 2% O₂) conditions.

After 48 h of incubation under normoxic and hypoxic conditions, the yeast cells or the CHO cells are collected and total RNA is isolated. The expression pattern of the respective SH2A-like genes under the different oxygen concentrations is analysed by northern blotting using labelled probes to the respective SH2A-like mRNAs or by quantitative RT-PCR.

### EXAMPLE 9

### Modulation of Yeast (Saccharomyces cerevisiae) SH2A-like Gene Expression by Gene Disruption and Overexpression; Analysis of Hypoxia Tolerance

To obtain mutant yeast with a knock out of the SH2A-like gene, an auxotrophic marker such as URA4, is cloned in between two fragments of the yeast SH2A-like gene. Said fragments are obtained by PCR using two sets of primers, a first to amplify a 5′ part of the SH2A-like gene and a second to amplify a 3′ part of the SH2A-like gene. The resulting SH2A-like gene fragment disrupted by URA4 is used to transform an uracil prototrophic yeast strain. Stable ura+ transformants, i.e. those in which the disrupted SH2A-like gene fragment has homologously recombined with the endogenous gene, are selected and analysed by PCR to affirm the disruption of the yeast SH2A gene homolog.

Selected yeast strains are subsequently analysed for tolerance to hypoxia by determining the growth rate relative to the growth rate of the corresponding wild-type yeast and under incubation conditions as described in Example 8. Mutants less tolerant to hypoxia than wild-type yeast can furthermore be transformed with SH2A-like genes of different sources to analyse complementation of the knocked out yeast SH2A-like gene.

For overexpression of the yeast SH2A gene homolog, the coding region of said gene is operably linked to a constitutive promoter such as the PGK-promoter or to an inducible promoter such as the ADH2-promoter. The resulting chimeric gene is introduced into an autonomously replicating plasmid such as the 2μ-plasmid. The plasmid carrying the chimeric SH2A gene is transformed to yeast applying the appropriate selection. Transformed yeast strains are subsequently analysed, if necessary after induction of expression of the chimeric SH2A gene, for increased tolerance to hypoxia by determining growth rates relative to the growth rate of an untransformed yeast and under the incubation conditions as described in Example 8.

### EXAMPLE 10

### Modulation of Arabidopsis thaliana SH2A-homologous Gene Expression: Transgenic Plants (Over)expressing an A. thaliana SH2A Gene Homolog in Sense and Antisense Orientation

Agrobacterium tumefaciens is used to transform A. thaliana following the floral dip transformation method (Clough and Bent 1998, Plant J. 16:735-743) or another suitable transformation method. The plant transformation vector contained within A. tumefaciens harbours a T-DNA carrying either:

a) For overexpression of a sense SH2A gene homolog: a plant selectable marker such as the kanamycin resistance marker and an A. thaliana SH2A gene homolog operably linked to a constitutive promoter such as the CaMV 35S or a tissue-specific or tissue-preferred promoter to obtain meristem-specific expression for SH2A; or

b) For antisense suppression of SH2A gene homologs: a plant selectable marker such as the kanamycin resistance marker and an A. thaliana SH2A gene homolog, or part thereof, linked in antisense orientation to a constitutive, tissue-specific or tissue-preferred promoter, preferably a meristem specific promoter for SH2A.

Selected transformed A. thaliana plants are selfed at flowering and homozygous progeny is identified and further analyzed as described in Example 15.

### EXAMPLE 11

### Overexpression and Suppression of Oryza sativa SH2A and SH2B Gene Expression in Transgenic Rice

Agrobacterium tumefaciens is used to transform embryogenic callus derived from immature embryos of O. sativa (Hiei et al. 1994, Plant J. 6: 271-282). The plant transformation vector contained within A. tumefaciens harbours a T-DNA carrying either:

a) For overexpression of the sense SH2A or SH2B gene: a plant selectable marker such as the nptII gene and the rice SH2A and SH2B gene operably linked to a constitutive promoter or preferably to a meristem-specific promoter for SH2A; or

b) For suppression of SH2A and SH2B gene expression: a plant selectable marker such as the nptII gene and the rice SH2A and SH2B gene, or part thereof, linked in sense and antisense orientation to a constitutive promoter or, in case of the SH2A gene preferably to a meristem-specific promoter.

A constitutive promoter may be used to obtain gene expression or suppression in all tissues or a tissue-preferred or tissue-specific promoter may be used to obtain gene expression or suppression in certain tissues only such as in meristems in case of SH2A.

Selected transformed O. sativa plants are selfed and single-locus transformants are identified for phenotypic studies (see Example 15).

### EXAMPLE 12

### Analysis of Tolerance to Hypoxia of A. thaliana Plants Displaying Modulated SH2A-like Gene Expression

The homozygous A. thaliana plants with modulated expression of a SH2A gene homolog obtained in Example 10 are analysed for hypoxia tolerance by assessing their tolerance to flooding. Parameters for flooding tolerance are:

a) plant survival rate

b) size of the surviving plants

c) flowering time of the surviving plants.

### EXAMPLE 13

### Overexpression of a SH2A-like Gene in Animal Cells and Analysis of Hypoxia Tolerance

The coding sequence of a human or mouse SH2A gene homolog is cloned in a selectable mammalian expression vector such as pcDNA3.1 (In Vitrogen, CA, USA) containing the cytomegalovirus (CMV) enhancer-promoter for high-level expression and containing the neo gene for selection of transfected cells. The resulting vector is transfected into CHO cells (see Example 8 for culture maintenance) by the use of Lipofectamine (Life Technologies, MD, USA) following the protocol of the manufacturer. Neomycin-resistant colonies are picked up and plated at a density of 5×10⁴ cells in a 24-well plate (Nunc, Denmark) and cultured for 12 h under normoxic conditions (see Example 8) after which the medium is refreshed. Untransformed cells are inoculated and incubated concurrently under the same conditions.

After refreshing the medium, plates with transformed cells expressing the SH2A gene homolog and untransformed cells are incubated under hypoxic conditions (2% oxygen) for different time periods (48 h, 72 h and 96 h). Survival rate of the different cells after the different incubation times is assessed by viability staining using the trypan blue exclusion method and/or the neutral red uptake method. Dead cells are stained by incubation with trypan blue (400 mg/L in PBS) for 10 min and can be observed microscopically. Living cells are stained by incubation with neutral red (56 mg/L in PBS) for 2 h. After washing with PBS, the cells are lysed in acidic EtOH (100 mM sodium citrate pH 4, 50% EtOH) and release of the dye taken up by viable cells is quantified by absorbance measurement at 540 nm.

### EXAMPLE 14

### Modulation of Anaerobic Response Gene Expression by Modulation of SH2A-like Gene Expression

Yeast incubated under hypoxic conditions and submerged A. thaliana plants displaying modulated expression of SH2A-like genes (see Examples 9-10) are compared with wild-type yeast incubated under hypoxic conditions and submerged wild-type A. thaliana plants, respectively, for the expression pattern of the gene encoding the anaerobic response protein pyruvate decarboxylase.

### EXAMPLE 15

### Phenotypic Analysis of O. sativa and A. thaliana Transgenic Plants Displaying Modulated Gene Expression of the SH2A and SH2B Genes or Homologs

The transgenic O. sativa and A. thaliana plants with modulated expression of a SH2A or SH2B gene or a homolog obtained in Examples 10 and 11 are analysed for hypoxia tolerance by assessing their tolerance to flooding. Parameters for flooding tolerance are:

a) plant survival rate

b) size of the surviving plants

c) flowering time of the surviving plants.

Enhanced survival and increased biomass are observed for plants transformed with SH2A or SH2B or homologs as compared to non transgenic controls.

In addition, general yield-related parameters are also analyzed under normal growth conditions.

### EXAMPLE 16

### Identification of Transposon Insertion Mutants in the ATH3 Gene of A. thaliana

A collection of En-1 transposon insertion mutants of A. thaliana (ZIGIA collection, Max-Planck-Institut für züchtungsforschung, Cologne, Germany) was screened for insertions in the ATH3 gene following a PCR-based approach and four independent lines were identified. Attempts to produce homozygous insertion mutants from these 4 lines failed. These results indicate that ATH3 knockouts are not viable and thus that ATH3 is an essential gene in A. thaliana. Similar screenings are conducted for the A. thaliana genes ATH1, ATH2, and ATH4.

### REFERENCES

1. WO98/36083 Baulcombe D. C. and Angell S. M. Methods and means for gene silencing in transgenic plants.

2. WO98/53083 Grierson D., Lowe A. L. and Hamilton A. J. Gene silencing.

3. WO99/15682 Baulcombe D. C., Voinnet O., and Lederer C. W. Gene silencing materials and methods.

4. WO99/53050 Waterhouse P. M., Wang M. B., and Graham M. W. Methods and means for obtaining modified phenotypes.

5. Altschul, S. F., Madden, T. L., Schaffer, A. A., Zhang, J., Zhang, Z., Miller, W., Lipman, D. J. (1997). Gapped BLAST and PSI-BLAST: a new generation of protein database search programs. Nucl. Acids. Res. 25: 3389-3402.

6. Bairoch, A., Apweiler, R. (1998). The SWISS-PROT protein sequence data bank and its supplement TrEMBL in 1998. Nucl. Acids. Res. 26(1): 38-42.

7. Barker, W. C., Garavelli, J. S., Haft, D. H., Hunt, L. T., Marzec, C. R., Orcutt, B. C., Srinivasarao, G. Y., Yeh, L. -S. L., Ledley, R. S., Mewes, H. -W., Pfeiffer, F., Tsugita, A. (1998). The PIR-international protein sequence database. Nucl. Acids. Res. 26(1): 27-32.

8. Bell M. H., Halford N. G., Ormrod J. C., Francis D. 1993. Tobacco plants transformed with cdc25, a mitotic inducer gene from fission yeast. Plant Mol.Biol. 23:445-451.

9. Benson, D. A., Boguski, M. S., Lipman, D. J., Ostell, J., Ouellette, B. F. (1998). GenBank. Nucl. Acids. Res. 26(1): 1-7.

10. Binarova P, Dolezel J, Draber P, Heberle-Bors E, Strnad M, Bogre L. 1998. Treatment of Vicia faba root tip cells with specific inhibitors to cyclin-dependent kinases leads to abnormal spindle formation. Plant J 16:697:707.

11. Bögre, L., Zwerger, K., Meskiene, I., Binarova, P., Csizmadia, V., Pongor, S., Wagner, E., Hirt, H., and Heberle-Bors, E. The cdc2Ms Kinase Is Differently regulated in the cytoplasm and in the nucleus. Plant Physiol 113, 841-852. 1997.

12. Bögre, L., Calderini O., Binarova P., Mattauch M, Till S., Kiegerl S., Jonak C., Pollaschek C., Barker P., Huskisson S., Hirt H., Heberle-Bors, E. 1999. A MAP Kinase Is Activated Late in Plant Mitosis and Becomes Localized to the Plane of Cell Division. Plant Cell 11:101-114.

13. Buchanan-Wollaston V., Ainsworth, C. (1997). Leaf senescence in Brassica napus: cloning of senescence related genes by subtractive hybridization. Plant. Mol. Biol. 33: 821-834.

14. Calderini O., Bögre L., Vicente O., Binarova P., Heberle-Bors E., Wilson C. 1998. A cell cycle regulated MAP kinase with a possible role in cytokinesis in tobacco cells. J.Cell Sci. 111:3091-3100.

15. Chou, P. Y., Fasman, G. (1978). Empirical predictions of protein conformation. Ann. Rev. Biochem. 47: 251-276.

16. Cohen-Fix O, Koshland D. 1997. The metaphase-to-anaphase transition:avoiding a mid-life crisis. Curr Opin Cell Biol 9:800-806.

17. Colasanti J., Tyers M., Sundaresan, V. 1991. Isolation and characterization of cDNA clones encoding a functional p34cdc2 homologue from Zea mays. Proc.Natl.Acad.Sci.U.S.A. 88:3377-3381.

18. Dellaporta, S. L., Wood, J., Hicks, J. B. (1983). A plant DNA minipreparation: version II. Plant Mol. Biol. Rep. 1(4): 19-21.

19. De Veylder L, Seger G, Glab N, Casteels P, Van Montagu M, Inze D. 1997. The Arabidopsis Cks1At protein binds the cyclin-dependent kinases Cdc2aAt and Cdc2bAt, FEBS Lett 412:446-452.

20. Doerner P., Jorgensen J. E., You R., Steppuhn J., Lamb, C., 1996. Control of root growth and development by cyclin expression. Nature 380:520-523.

21. Dolferus, R., Jacobs, M., Peacock, W. J., Dennis, E. S. (1994). Differential interactions of promoter elements in stress responses of the Arabidopsis Adh gene. Plant Physiol. 105:1075-1087.

22. Elledge S J. 1996. Cell Cycle checkpoints: preventing an identity crises. Science 274:1664-1672.

23. Evans, T., Rosenthal E. T., Youngblom J., Distel D., HUNT T. 1983. Cyclin:a protein specified by maternal mRNAA in sea urchin eggs that is destroyed at each cleavage division. Cell 33:389-396.

24. Fantes P. 1989. Yeast cell cycle. Curr.Opin.Cell Biol. 1:250-255.

25. Feiler H S, Jacobs T W. 1990. Cell division in higher plants:a cdc2 gene, its 34-kDa product, and histone H1 kinase activity in pea. Proc Natl Acad Sci USA 87:5397-5401.

26. Fesquet D., Labbe J.C., Derancourt J., Capony J. P., Galas S., Girard F., Lorca T., Shuttleworth J., Doree M., Cavadore J. C. 1993. The MO15 gene encodes the catalytic subunit of a protein kinase that activates cdc2 and other cyclin-dependent kinases (CDKs) through phosphorylation of Thr161 and its homologues. EMBO J. 12:3111-3121.

27. Francis D., Halford N. G. 1995. The plant cell cycle. Physiologia Plantarum 93, 365374.

28. Francis D., Dudiits D. E., Inzé, D. 1998. Plant cell division. Portland, London.

29. Gallegos, M. T., Schleif, R., Bairoch, A., Hofmann, K., Ramos, J. L. (1997). The Arac/XylS family of transcriptional regulators. Microbiol. Mol. Bio. Rev. 61(4): 393-410.

30. Gatz, C., Frohberg, C., Wendenburg R. (1992). Stringent repression and homogeneous de-repression by tetracycline of a modified CaMV 35S promoter in intact tobacco plants. Plant J. 2:397-404.

31. Gibrat, J. -F., Gamier, J., Robson, B. (1987). Further development of protein secondary structure prediction using information theory. New parameters and consideration of residue pairs. J. Mol. Biol. 198: 425-443.

32. Glotzer, M., Murray, A. W., Kirschner, M. W. (1991). Cyclin is degraded by the ubiquitin pathway. Nature 349: 132-138.

33. Gubler, F., Kalla, R., Roberts, J. K., Jacobsen, J. V. (1995). Gibberellin-regulated expression of a myb gene in barley aleurone cells: evidence for Myb transactivation of a high-pI alpha-amylase promoter. Plant Cell 7:1879-1891.

34. Gubler, F., Jacobsen, J. V. (1992). Gibberellin-responsive elements in the promoter of a barley high-pI alpha-amylase gene. Plant Cell 4:1435-1441.

35. Gucuyener, K., Atalay, Y., Aral, Y. Z., Hasanoglu, A., Turkyilmaz, C., Biberoglu, G. (1999). Excitatory amino acids and taurine levels in cerebrospinal fluid of hypoxic ischemic encephalopathy in newborn. Clin. Neurol. Neurosurg. 10 1: 171-174.

36. Hayles J., Nurse P. 1986. Cell cycle regulation in yeasst. J.Cell Sci.Suppl 4:155-170.

37. Hemerly A. S., Ferreira P., De Almeida E. J., Van Montagu M., Engler G., Inze D. 1993. cdc2a expression in Arabidopsis is linked with competence for cell division. Plant Cell 5:1711-1723.

38. Hirt H., Pay A., Gyorgyey J., Bako L., Nemeth K., Bögre L., Schweyen R. J., Heberle-Bors E., Dudits D. 1991. Complementation of a yeast cell cycle mutant by an alfalfa cDNA encoding a protein kinase homologous to p34cdc2. Proc.Natl.Acad.Sci.U.S.A. 88:1636-1640.

39. Hochstrasser M. 1998. There's the rub:a novel ubiquitin-like modification linked to cell cycle regulation. Genes Dev 12:901-907.

40. Huntley R, Healy S, Freeman D, Lavender P, de Jager S, Greenwood J, Makker J, Walker E, Jackman M, Xie Q, Bannister AJ, Kouzaarides T, Gutierrez C, Doonan J H, Murray JA. 1998. The maize retinoblastoma protein homologue ZmRb-1 is regulated during leaf development and displays conserved interactions with G1/S regulators and plant cyclin D (CycD) proteins. Plant Mol Biol 37:155-169.

41. Ito, M., Matsuo, Y., Nishikawa, K. (1997). Prediction of protein secondary structure using the 3D-1D compatibility algorithm. CABIOS 13: 415-423.

42. John, P. C. L. 1981. The Cell cycle. Cambridge University Press, Cambridge Cambridge shire.

43. John, P. C., Sek F. J., Lee M. G. 1989. A homolog of the cell cycle control protein p34cdc2 participates in the division cycle of Chlamydomonas, and a similar protein is detectable in higher plants and remote taxa. Plant Cell 1:1185-1193.

44. Kneller, D. G., Cohen, F. E., Langridge, R. (1990). Improvements in protein secondary structure prediction by enhanced neural network. J. Mol. Biol. 214: 171-182.

45. Krek W. 1998. Proteolysis and the G1-S transition: the SCF connection. Curr.Opin.Genet.Dev. 8:36-42.

46. Kumagai A., Dunphy W. G. 1991. The cdc25 protein controls tyrosine dephosphorylation of the cdc2 protein in a cell-free system. Cell 64:903-914.

47. Kyte, J., Doolittle, R. F. (1982). A simple method for displaying the hydropathic character of a protein. J. Mol. Biol. 157: 105-132.

48. Labbe J.C., Capony J.P., Caput D., Cavadore J.C., Derancourt J., Kaghad M., Lelias J. M., Picard A., DOREE M. 1989. MPF from starfish oocytes at first meiotic metaphase is a heterodimer containing one molecule of cdc2 and one molecule of cyclin B. EMBO J. 8:3053-3058.

49. Lake R. S., Salzman N. P. 1972. Occurrence and properties of a chromatin-associated F1-histone phosphokinase in mitotic Chinese hamster cells. Biochemistry 11:4817-4826.

50. Langan T. A. 1978. Methods for the assessment of site-specific histone phosphorylation. Methods Cell Biol. 19:127-142.

51. Lee M. G., Nurse P. 1987. Complementation used to clone a human homologue of the fission yeast cell cycle control gene cdc2. Nature 327:31-35.

52. Lisztwan J, Marti A, Sutterluty H, Gstaiger M, Wirbelauer C, Krek W. 1998. Association of human CUL-1 and ubiquitin-conjugating enzyme CDC34 with the F-box protein p45(SKP2):evidence for evolutionary concervation in the subunit composition of the CDC34-SCF pathway, EMBO J 17:368-383.

53. Lorbiecke, R., Sauter, M. (1997). A ribosomal 5S rRNA-binding protein gene from rice (Oryza sativa L.) is regulated in a cell cycle phase-specific manner and in response to gibberellin. J. Plant Physiol. 151: 334-338.

54. Lorbiecke, R., Sauter, M. (1999). Adventitious root growth and cell-cycle induction in deepwater rice. Plant Physiol. 119: 21-29.

55. Lundgren K, Walworth N, Booher R, Dembski M, Kirschner M, Beach D. 1991. mikl and weel cooperate in the inhibitory tyrosine phosphorylation of cdc2. Cell 64:1111-1122.

56. Manjunath, S., Sachs, M. M. (1997). Molecular characterization and promoter analysis of the maize cytosolic glyceraldehyde 3-phosphate dehydrogenase gene family and its expression during anoxia. Plant Mol. Biol. 33:97-112.

57. Maruyama, S., Hirayama, C., Oyake, N., Kadowaki, Y., Umeki, K., Sagayama, A., Kato, K., Fukuda, K., Kuzuo, H., Ohuchi, Y. (1999). Am. J. Gastroenterol. 94:2994-2999.

58. Masuda, S., Moon, S. K., Kambe, T., Nagao, M., Sasaki, R. (2000). A new biological strategy for high productivity of recombinant proteins in animals cells by the use of hypoxia-response enhancer. Biotechnol. Bioeng. 67, 157-164.

59. Mokhashi, M. S. (1999). Hypoxaemia-think of the liver! Every internist should be aware of the hepatopulmonary syndrome. Postgrad. Med. J. 75:295-297.

60. Murray A. W., Kirschner M. W. 1989. Dominoes and clocks:the union of two views of the cell cycle. Science 246:614-621.

61. Naldini, A., Carraro. F. (1999). Hypoxia modulates cyclin and cytokine expression and inhibits peripheral mononuclear cell proliferation. J. Cell. Physiol. 181:448-454.

62. Nasmyth K. 1993. Control of the yeast cell cycle by the Cdc28 protein kinase. Curr. Opin. Cell Biol. 5:166-179.

63. Nicholas, K. B., Nicholas, H. B. Jr. (1997). GeneDoc: a tool for editing and annotating multiple sequence alignments. Distributed by the author.

64. Norbury C., Nurse, P. 1992. Animal cell cycles and their control. Annu.Rev.Biochem. 61:441-470.

65. Norman, J. T., Ophanides, C., Garcia, P., Fine, L. G. (1999). Hypoxia-induced changes in extracellular matrix metabolism in renal cells. Exp. Nephrol. 7:463-469.

66. Nurse P. 1990. Universal control mechanism regulating onset of M-phase. Nature 344:503-508.

67. Nurse P., Bissett Y. 1981. Gene required in GI for commitment to cell cycle and in G2 for control of mitosis in fission yeast. Nature 292:558-560.

68. Ohme-Takagi, M., Shinshi, H. (1995). Ethylene-inducible DNA binding proteins that interact with an ethylene-responsive element. Plant Cell 7:173-182.

69. Olive, M. R., Walker, J. C., Singh, K., Dennis, E. S., Peacock, W. J. (1990). Functional properties of the anaerobic responsive element of the maize Adhl gene. Plant Mol. Biol. 15:593-604.

70. Ormrod J.C., Francis, D. 1993. Molecular and cell biology of the plant cell cycle proceedings of a meeting held at Lancaster University, Apr. 9-10, 1992. Kluwer Academic, Dordrecht.

71. Page, R. D. M. (1996). TREEVIEW: An application to display phylogenetic trees on personal computers. Computer Applications in the Biosciences 12: 357-358.

72. Pines J. 1995. Cell cycle. Confirmational change [news; comment]. nature 376:294-295.

73. Poon R. Y., Ramashita K., Adamczewski J. P., Hunt T., Shuttleworth J. 1993. The cdc2-related protein p40MO15 is the catalytic subunit of a protein kinase that can activate p33cdk2 and p34cdc2. EMBO J. 12:3123-3132.

74. Raskin, I., Kende, H. (1984). Regulation of growth in stem sections of deep-water rice. Planta 160: 66-72.

75. Reed S. I., Hadwiger J. A., Lorincz A. T. 1985. Protein kinase activity associated with the product of the yeast cell division cycle gene CDC28. Proc.Natl.Acad.Sci.U.S.A. 82:4055-4059.

76. Renaudin J P, Doonan J H, Freeman D, Hashimoto J, Hirt H, Inze D, Jacobs T, Kouchi H, Rouze P, Sauter M, Savoure A, Sorrell DA, Sundaresan V, Murray J A. 1996. Plant cyclins:a unified nomenclature for plant A-, B- and D-type cyclins based on sequence organization. Plant Mol Biol 32:1003-1018.

77. Rost, B., Sander, C. (1994). Combining evolutionary information and neural networks to predict protein secondary structure. Proteins 19: 55-72.

78. Russell P, Nurse P. 1986. cdc25+functions as an inducer in the mitotic control of fission yeast. Cell 45:145-153.

79. Russell P., Nurse P. 1987b. Negative regulation of mitosis by wee1+, a gene encoding a protein kinase homolog. Cell 49:559-567.

80. Russell P., Nurse P. 1987a. The mitotic inducer nim1+ functions in a regulatory network of protein kinase homologs controlling the initiation of mitosis. Cell 49:569-576.

81. Sanger, F., Nickler, S., Coulson, A. R. (1977). DNA sequencing with chain-terminating inhibitors. Proc. Natl. Acad. Sci. USA 74: 5463-5467.

82. Sauter M. (1997). Differential expression of a CAK (cdc2-activating kinase)-like protein kinase, cyclins and cdc2 genes from rice during the cell cycle and in response to gibberellin. The Plant Journal 11 (2): 181-190.

83. Sauter, M., Mekhedov, S. L., Kende, H. (1995). Gibberellin promotes histone H1 kinase activity and the expression of cdc2 and cyclin genes during the induction of rapid growth in deepwater rice intemodes. Plant J. 7(4): 623-632.

84. Setter, T. L., Ellis, M., Laureles, E. V., Ella, E. S. Senadhira, D., Mishra, S. B., Sarkarung, S., Datta, S. (1997) Physiology and genetics of submergence tolerance in rice. Annal. Bot. 19(Supp.A):67-77.

85. Sinusas, A. J. (1999). The potential for myocardial imaging with hypoxia markers. Semin. Nuel. Med. 29:330-338.

86. Soni R, Carmichael J P, Shah Z H, Murray J A. 1995. A family of cyclin D homologs from plants differentially controlled by growth regulators and containing the conserved retinoblastoma protein interaction motif. Plant Cell 7:85-103.

87. Sorrell D A, Combettes B, Chaubet-Gigot C, Murray J A. 1999. Distinct cyclin D genes show mitotic accumulation or constant levels of transcripts in tobacco bright yellow-2 cells. Plant Physiol 119:343-352.

88. Steele, M. I., Lorenz, D., Hatter, K., Park, A., Sokatch, J. R. (1992). Characterization of the mmsAB operon of Pseudomonas aeruginosa encoding methylmalonate-semialdehyde dehydrogenase and 3-hydroxyisobutyrate dehydrogenase. J. Biol. Chem. 267(19): 13585-13592.

89. Stoesser, G., Moseley, M. A., Sleep, J., McGowran, M., Garcia-Pastor, M., Sterk, P. (1998). The EMBL nucleotide sequence database. Nucl. Acids. Res. 26(1): 8-15.

90. Sun Y, Kilkes B P, Zhang C, Dante R A, Carneiro N P, Lowee K S, Jung R, Gordon-Kamm W J, Karkins B A 1999. Characterization of maize (Zea mays L.) Wee1 and its activity in developing endosperm. Proc Natl Acad Sci U S A 96:4180-4185.

91. Swenson K. I., Farrell K. M., Ruderman J. V. 1986. The clam embryo protein cyclin A induces entry into M phase and the resumption of meiosis in Xenopus oocytes. Cell 47:861-870.

92. Tateno, Y., Fukami-Kobayashi, K., Miyazaki, S., Sugawara, H., Gojobori, T. (1998). DAN data bank of Japan ta work on genome sequence data. Nucl. Acids. Res. 26(1): 16-20.

93. Thomson, J. D., Higgins, D. G., Gibson, T. J. (1994). CLUSTAL W: improving the sensitivity of progressive multiple sequence alignment through sequence weighting, positions-specific gap penalties and weight matrix choice. Nucl. Acids. Res. 22: 4673-4680.

94. Wang, H. and Crosby, W. L. A plant cyclin-dependent kinase inhibitor gene. nature 386, 451-452. 1997.

95. White, J. V., Stultz, C. M., Smith, T. F. (1994). Protein classification by stochastic modeling and optimal filtering of amino-acid sequences. Mathematical Biosciences 119: 35-75.

96. Wiedman, M., Tabin, G. C. (1999). High-altitude retinopathy and altitude illness. Ophthalmology 106:1924-1926.

97. Wilson C., Pfosser M., Jonak C., Hirt H., Heberle-Bors E., Vicente O. 1999. Evidence for the activation of a MAP kinase upon phosphate-induced cell cycle re-entry in tobacco cells. Physiol.Plant 102:532-538.

98. Xie Q, Sanz-Burgos A P, Hannon G J, Gutierrez C. 1996. Plant cells contain a novel member of the retinoblastoma family of growth regulatory proteins. EMBO J 15:4900-4908.

99. Xu, K., Mackill, D. J. (1996). A major locus for submergence tolerance mapped on rice chromosome 9. Mol. Breeding 2:219-224.

100. Zaman, K., Ryu, H., Hall, D., O→Donovan, K., Lin, K. I., Miller, M. P., Marquis, J. C., Baraban, J. M., Semenza, G. L., Ratan, R. R. (1999). Protection from oxidative stress-induced apoptosis in cortica neuronal cultures by iron chelators is associated with enhanced DNA binding of hypoxia-inducible factor-1 and ATF-1/CREB and increased expression of glycolytic enzymes, p21(waf1/cip1), and erythropoietin. J. Neurosci. 19:9821-9830.

101. Zeng Y, Forbes KC, Wu Z, Moreno S, Piwnica-Worms H, Enoch T. 1998. Replication checkpoint requires phosphorylation of the phosphatase Cdc25 by Cds1 or Chk1. nature 395:507-510.

102. Zhong, H., De Marzo, A. M., Laughnier, E., Lim, M., Hilton, D. A., Zagzag, D., Buechler, P., Isaacs, W. B., Semenza, G. L., Simons, J. W. (1999). Overexpression of hypoxia-inducible factor 1 alpha in common human cancers and their metastases. Cancer Res. 59:5830-5835.

What is claimed is:

1. A transgenic plant, an essentially derived variety thereof, a plant part, or plant cell which comprises a nucleotide sequence for an SH2A or SH2A-like gene wherein said nucleotide sequence is heterologous to the genome of said transgenic plant, essentially derived variety thereof, plant part or plant cell.

2. A transgenic plant, an essentially derived variety thereof, a plant part, or plant cell which comprises a nucleotide sequence for an SH2A or SH2A-like gene wherein said nucleotide sequence has been introduced into the transgenic plant, plant part or plant cell by recombinant DNA means.

3. A transgenic plant, an essentially derived variety thereof, a plant part, or plant cell which comprises an SH2A or SH2A-like protein wherein said SH2A or SH2A-like protein is heterologous to the transgenic plant, essentially derived variety thereof, plant part or plant cell.

4. A plant cell or protoplast transformed with a nucleotide sequence for an SH2A or SH2A-like gene wherein said nucleotide sequence is heterologous to the genome of the plant cell or plant protoplast.

5. The plant cell or protoplast of claim 4 wherein the plant cell is stably or transiently transformed.

6. A host cell which comprises a nucleotide sequence for an SH2A or SH2A-like gene wherein said nucleotide sequence is heterologous to the genome of said host cell or wherein said nucleotide sequence has been introduced into said host cell by recombinant DNA means.

7. A host cell according to claim 6 wherein said host cell is a bacterial, yeast, fungal, insect, plant, animal or human cell.

8. A host cell according to claim 6 wherein the nucleotide sequence for an SH2A or SH2A-like gene is in the sense or antisense orientation relative to a regulatory region directing expression of said nucleotide sequence or wherein said nucleotide sequence is included in a gene silencing construct driven by a regulatory region.

9. A method for modulating growth or survival of cultured cells under hypoxic conditions which comprises modulating the level and/or activity of an SH2A or SH2A-like protein in said cultured cells.

10. A method for altering growth response in cultured cells which comprises modulating the level and/or activity of an SH2A or SH2A-like protein in said cultured cells.

11. The method of claim 9 or 10 wherein the cultured cells are bacterial, yeast or fungal cells.

12. The method of claim 9 or 10 wherein the cultured cells are animal, human or insect cells.

13. The method of claim 9 or 10 wherein the cultured cells are plant cells.

14. A method for altering growth response in cells, tissues or organs of an organism which comprises modulating the level and/or activity of an SH2A or SH2A-like protein in said cells, tissues or organs of said organism.

15. A method for altering growth response in cells, tissues or organs of a plant which comprises modulating the level and/or activity of an SH2A or SH2A-like protein in said cells, tissues or organs of said plant.

16. The method according to claim 15 wherein the level of SH2A or SH2A-like protein is modulated by increasing transcription of a nucleotide sequence for said SH2A or SH2A-like protein.

17. The method according to claim 16 wherein the increase in transcription is induced by exposing the cells, tissues or organs of a plant to ethephon or ethylene.

18. A method for producing a plant which is adapted to growth in hypoxic conditions which comprises transforming at least one of a plant cell, pollen, protoplast, explant, plant part or plant organ with a coding sequence for an SH2A or like gene and regenerating a plant therefrom.

19. A method for improving survival of a plant in conditions of low oxygen which comprises transforming at least one of a plant cell, pollen, protoplast, explant, plant part or plant organ with a coding sequence for an SH2A or SH2A-like gene and regenerating a plant therefrom.

20. A method for improving water logging tolerance in a plant which comprises transforming at least one of a plant cell, pollen, protoplast, explant, plant part or plant organ with a coding sequence for an SH2A or SH2A-like gene and regenerating a plant therefrom.

21. A method for inducing gibberellin biosynthesis in a plant cell, protoplast, explant, plant part or plant organ, said method comprising modulating the level and/or activity of SH2A or SH2A-like protein therein.

22. A method for inducing gibberellin biosynthesis in a plant, said method comprising modulating the level and/or activity of SH2A or SH2A-like protein in the cells or a group of cells of said plant.

23. A method of regulating an anaerobic response protein in a plant cell, protoplast, explant, plant part or plant organ which comprises modulating the level and/or activity of an SH2A or SH2A-like protein therein.

24. The method of claim 23 wherein the anaerobic response protein is pyruvate decarboxylase 2.

25. A genetic construct comprising a nucleotide sequence for an SH2A or SH2A-like gene operably linked to a promoter sequence which directs expression of said nucleotide sequence.

26. The genetic construct of claim 25 wherein the SH2A or SH2A-like gene is a cDNA or genomic sequence.

27. The genetic construct of claim 25 wherein the SH2A or SH2A-like gene is a synthetic sequence.

28. The genetic construct of any one of claims 25-27 wherein the nucleotide sequence for an SH2A or SH2A-like gene is in a sense or antisense orientation relative to the promoter sequence or is included in a gene silencing construct.

29. A chimeric gene construct comprising a gene coding for an SH2A or SH2A-like protein wherein said gene is under the control of a promoter which functions in plants.

30. A chimeric gene construct comprising an SH2A or S142A-like gene promoter operably linked to a heterologous coding sequence.

31. An isolated nucleic acid coding for an SH2A-like protein selected from the group consisting of nucleic acid sequences set forth in SEQ ID NOs:5. 7, 9, 11, 13, 15, and 17.

32. An isolated SH2A-like protein having an amino acid sequence selected from the group consisting of amino acid sequences set forth in SEQ ID NO:6, 8, 10, 12, 14, 16 and 18.

33. Pollen from the transgenic plant or essentially derived variety thereof of any one of claims 1-3.

34. Seed from the transgenic plant or essentially derived variety thereof of any one of claims 1-3.

35. A cutting from the transgenic plant or essentially derived variety thereof of any one of claims 1-3.

36. A flower from the transgenic plant or essentially derived variety thereof of any one of claims 1-3.


SEQUENCE LISTING (18 sequences)

SEQ ID NO: 1 - DNA, 872 bp, Rice
  CDS (69)..(668)
agacgaacaaaaaacagaatccatcgccataatcgaaggttcgctcttgcttccaccccgcaatccacatggagaacgaattccaggatggtaagacggaggtgatagaagcatggtacatggatgatagcgaagaggaccagaggcttcctcatcaccgcgaacccaaagaattcattcctgttgataagcttacagaactaggagtaatcagctggcgcctaaatcctgataactgggagaattgcgagaacctgaagagaatccgcgaagccagaggttactcttatgtggacatttgtgatgtgtgcccagagaagctgccaaattatgaaactaagatcaagagtttctttgaagaacacctgcataccgatgaagaaatacgctattgtcttgaagggagtggatactttgatgtgagagaccaaaatgatcagtggattcgtatagcactgaagaaaggaggcatgattgttctgcctgcagggatgtaccaccgctttacgttggacaccgacaactatatcaaggcaatgcgactgtttgttggcgatcctgtttggacaccctacaaccgtccccatgaccatcttcctgcaagaaaggagtttttggctaaacttctcaagtcagaaggtgaaaatcaagcagttgaaggcttctgagggttttgttgggctcctgcactgcggttctatattcaacctgaataagatgtgctatagcaatgtaaatttagcacagtggctatggtcgccactcaccaacttgaagtgaaagatttaatgatttttgttaattcttatgtatcaatcggcatatagcatttccgaaatgtgttttcaataaacaggagtcatgaagctgaa

SEQ ID NO: 2 - protein, 199 aa, Rice
MENEFQDGKTEVIEAWYMDDSEEDQRLPHHREPKEFIPVDKLTELGVISWRLNPDNWENCENLKRIREARGYSYVDICDVCPEKLPNYETKIKSFFEEHLHTDEEIRYCLEGSGYFDVRDQNDQWIRIALKKGGMIVLPAGMYHRFTLDTDNYIKAMRLFVGDPVWTPYNRPHDHLPARKEFLAKLLKSEGENQAVEGF

SEQ ID NO: 3 - DNA, 980 bp, Rice
  CDS (139)..(735)
cggacgcgtgggcagattgcgttgagctgaagctgttcgtgtgactcttctacaccttccaggctatccggaatcgggagggtttcccaataggaaagcaactcaggactcaggagcggcgtctgagaggtttcagagatggagaaccagttccaggatggcaaggaggaggtcatcgaagcttggtacatggatgacagtgaagaggaccagaggcttcctcatcatcgtgagcccaaagaattcattcctcttagcaaactttcagagttaggaatattaagctggcgcctgaatgctgatgactgggagaatgatgagaacctcaagaaaatccgtgaggccaggggatactcttacatggatatttgtgatgtgtgtccagaaaagctgccaaactatgaggctaagctgaaaaatttctttgaagaacacttgcatactgatgaagagatacgctattgtcttgagggaagtggatacttcgatgtcagggaccaaaatgatcagtggatccgtgtagcagtgaagaaagggggcatgattgttttgcctgcgggaatgtatcaccgcttcacattggacagtgacaactacatcaaggcaatgcggctctttgtgggagagcctgtctggacgccgtacaaccgtccccatgaccatctgccagctagaaaggagtatgtcgaaaaaattatcaacaggggtggaactcaagctgtcgaagctcgttaaaggcatatcaagatgtgcttcctagttcggtgttctgttacactctacagatactgaataaactgtgctatcagctgttgcaatgggctcctaccgacatcttacatcatttggcagtattttgcacaaacccgcttaaaatctccctgaaaatacgcacgtcaccatgtcagagtgtttatatacaataatgacacttcagtccacagtcagcaagggactaatgacaaaaaaaaaaaaaaaaa

SEQ ID NO: 4 - protein, 198 aa, Rice
MENQFQDGKEEVIEAWYMDDSEEDQRLPHHREPKEFIPLSKLSELGILSWRLNADDWENDENLKKIREARGYSYMDICDVCPEKLPNYEAKLKNFFEEHLHTDEEIRYCLEGSGYFDVRDQNDQWIRVAVKKGGMIVLPAGMYHRFTLDSDNYIKAMRLFVGEPVWTPYNRPHDHLPARKEYVEKIINRGGTQAVEAR

SEQ ID NO: 5 - DNA, 774 bp, Tomato
  CDS (1)..(591)
gcaccagatccaagagaggatgtcatacaggcatggtacatggatgacaacgatgaggaccagaggcttcctcatcaccgtgagccaaaggaatttgtgtctcttgacaagctggctgaacttggagtgctcagctggagacttgatgctgacaattatgagactgatgaggagttgaagaaaattcgggaagatcgtggatattcatacattgatttctgtgaggtttgccctgagaaactaccgaattacgaggagaaaatcaagaacttttttgaagaacacctgcacaccgacgaggagatccgttacgctgttgcaggaagtggttactttgatgtccgcgatgtgaatgagagctggattcgcgtctgggtaaagaaaggtggaatgattgttcttcctgctggaatctatcaccgcttcacgcttgattcaagcaactacattaaggcaatgcgtctctttgttggtgacccaatttggactccatacaatcgtccacatgatcatcttcccgcaaggcaagaatatgttgagacgtttgtcaacgcagatggcgctggtcgtgctgttaatgctgctgcttaaatcaactataggagaggaatttgaaatcgtactagattgtaataaatattaccatatggtggctttgctgttcttgatgtgtgccttactaagcatgtttaatgttgtattgtggcactaaataaatcaccccctatgggagattgattgtttatatgcaagtggaatttattatgtgatttt

SEQ ID NO: 6 - protein, 196 aa, Tomato
APDPREDVIQAWYMDDNDEDQRLPHHREPKEFVSLDKLAELGVLSWRLDADNYETDEELKKIREDRGYSYIDFCEVCPEKLPNYEEKIKNFFEEHLHTDEEIRYAVAGSGYFDVRDVNESWIRVWVKKGGMIVLPAGIYHRFTLDSSNYIKAMRLFVGDPIWTPYNRPHDHLPARQEYVETFVNADGAGRAVNAAA

SEQ ID NO: 7 - DNA, 603 bp, Tomato
  CDS (3)..(572)
aaatggcaatcgagtgtaaggcatggtttatggatgaaaattcagaagatcagcggctaccgcaccagaagaacccaccggagtttgtttcagtggagaaattagcagtaatcggagttttatactggaaattgaaccctaatgattacgagaacgatgaagaattgaaaaaaattcgtcaaagtagaggctacagctacatggacttgctggatttgtgccctgagaaggtggataactatgagcagaagttgaaaaatttctatacggagcacatacacgcagatgaggagatacgttactgtctggaagggagtggatattttgatgtgagagacaaggatgatcgctggattcgcatctggatgaaggccggtgatatgattgtcttgcctgctgggatttaccaccggttcaccctagatactgataactatgtcaagttgatgaggttgtttgtgggagagccggtgtggacgccttacaatcgaccacaagaagatcatccagcaaggaaggagtacatcaagagtgttactgaaagagtaggagtgcctcttacagcacactaagacatatttgagctttacaaacctgagagtg

SEQ ID NO: 8 - protein, 189 aa, Tomato
MAIECKAWFMDENSEDQRLPHQKNPPEFVSVEKLAVIGVLYWKLNPNDYENDEELKKIRQSRGYSYMDLLDLCPEKVDNYEQKLKNFYTEHIHADEEIRYCLEGSGYFDVRDKDDRWIRIWMKAGDMIVLPAGIYHRFTLDTDNYVKLMRLFVGEPVWTPYNRPQEDHPARKEYIKSVTERVGVPLTAH

SEQ ID NO: 9 - DNA, 889 bp, Soybean
  CDS (32)..(634)
cgaacccgtcgtagcagaaaaacttgtcaccatggtttcttccgacaaggatccacgagaggatgtccttcaagcctggtacatggatgatagtgatgaagatcaaagactcccccaccacaaagaacccaaggagtttgtctcgttggaccaacttgctgaacttggagtccttagctggaaactagatgctgataaccatgaaaatgatccagagctgaagaagattcgtgaagagcgtggttacacctacatggatgtttgtgaggtctgcccagaaaagttgccaaattatgaacagaaaatcaaaagcttctttgaagagcatcttcacactgatgaggagatccgcttttgtgctgctggaagtggctattttgatgttagggatcgcaatgaagcttggattcgtgtgtgggtcaagaaaggaggaatgatcatcttacctgccggaatttatcatcgctttacgctagatgagagcaactacattaaggctttgcgtttttttgttggtgagccagtttggactccatacaatcgtccaaatgaccatctccctgcaagacaacaatatgtcaaggattttgtggaaaaggatgttagcagccatgctgttgatgccaccgcgtaagatctggttctgcctaatcatagtaccacatgaaaaggaccaagactttgttgctaaagtaaggtttgaaaaaaagaaaataatggtgtctttaaataaagggtcctggcttgttatgccttgatgtaccctcgccagtgtttttgttgcctgtccctgtataaagattgcattgtattattattagaattgggtacagaataaacataagcataagttagcatgctgatgtatatttatgtaaaaaaaaataaa

SEQ ID NO: 10 - protein, 200 aa, Soybean
MVSSDKDPREDVLQAWYMDDSDEDQRLPHHKEPKEFVSLDQLAELGVLSWKLDADNHENDPELKKIREERGYTYMDVCEVCPEKLPNYEQKIKSFFEEHLHTDEEIRFCAAGSGYFDVRDRNEAWIRVWVKKGGMIILPAGIYHRFTLDESNYIKALRFFVGEPVWTPYNRPNDHLPARQQYVKDFVEKDVSSHAVDATA

SEQ ID NO: 11 - DNA, 933 bp, Cotton
  CDS (33)..(635)
atttttttttaatttgacggaaaaaaaaaactatgaccatgggttctgcagacaagagggaggaagttattcaggcatggtacatggatgatagtgatgaagatcagaggcttcctcatcaccgtgaacctaaggaatatgtatccttggataaacttgctgagcttggagtactcagctggcgattggatgctgataactatgaaaatgatgaagagttgaagaaaattcgtgaagaacgaggttactcctacatggacttctgcgaggtttgccctgagaagcttccaaattatgaggagaagataaaaaatttcttcgaagaacatattcatactgatgaggagatccgttactgtgtggcaggaagtggttattttgatgtacgggatcataatgataaatggattcgtgtgtgggtgaagaaaggaggcatgatagttttacctgctggaatttatcatcgctttactctggatacagacaactatattaaggcaatgcggctctttgttggtgatccaatttggactccgtacaatcgtccgcacgatcatcttcctgcaaggaaggagtatatcaagaactttttgcgggaggaaggtggtggccaagccgttgatgctgccgcataaaatcaacattcatctggtggtggccaagtcgttgatgctgccgcataaaatcagcattcatctctggtatcgtgtcttataaaatatgaaaccccggatttgtggtaataaataagtctaggcttgtctgcttttgatgcgtggatatggatcgttatggttgttgcttgctatatattgcctattccatatcgaaaattcgcaaacttgctatgtatttctacattttatgtgcttactaccagattggctcttaataatcaaagtttacataatatacatttcgtcgacgcggccg

SEQ ID NO: 12 - protein, 200 aa, Cotton
MTMGSADKREEVIQAWYMDDSDEDQRLPHHREPKEYVSLDKLAELGVLSWRLDADNYENDEELKKIREERGYSYMDFCEVCPEKLPNYEEKIKNFFEEHIHTDEEIRYCVAGSGYFDVRDHNDKWIRVWVKKGGMIVLPAGIYHRFTLDTDNYIKAMRLFVGDPIWTPYNRPHDHLPARKEYIKNFLREEGGGQAVDAAA

SEQ ID NO: 13 - DNA, 920 bp, Human
  CDS (1)..(564)
cgaacacggcacccgcactgcgcgtcagtggtgcaggcctggtatatggacgacgccccgggcacccgcggcaaccccaccgccccgaccccggccgcccagtgcgctggagcagctgcgcggctcggggtgctctactggaagctggatgctgacaaatatgagaatgatccagaattagaaaagatccgaagagagaggaactactcctggatggacatcataaccatatgcaaagataaactaccaaattatgaagaaaagattaagatgttctacgaggagcatttgcacttggacgatgagatccgctacatcctggatggcagtgggtacttcgatgtgagggacaaggaggaccagtggatccggatcttcatggagaagggagacatggtgacgctccccgcggggatctatcaccgcttcacggtggacgagaagaactacacgaaggccatgcggctgtttgtgggagaaccggtgtggacagcgtacaaccggcccgctgaccattttgaagcccgcgggcagtacgtgaaatttctggcacagaccgcctagcagtgctgcctgggaactaacacgtgcctcgtaaaggtccccaatgtaatgaactgagcagaaaattcaatcaactttctctttgcttttagaggatagccttgaggtagattatctttcctttgtaagattatttgatcagaatattttgtaatgaaaggatctagaaagcaacttggaagtgtaaagagtcaccttcattttctgtaactcaatcaagactggtgggtccatggccctgtgttagttcattgcattcaggttgagtcccaaatgaaagtttcatctcccgaaatgcagttccttagatgcccatctggacgtgaatgccgcgcctgcgtgtaagaaggtgcaat

SEQ ID NO: 14 - protein, 187 aa, Human
RTRHPHCASVVQAWYMDDAPGTRGNPTAPTPAAQCAGAAARLGVLYWKLDADKYENDPELEKIRRERNYSWMDIITICKDKLPNYEEKIKMFYEEHLHLDDEIRYILDGSGYFDVRDKEDQWIRIFMEKGDMVTLPAGIYHRFTVDEKNYTKAMRLFVGEPVWTAYNRPADHFEARGQYVKFLAQTA

SEQ ID NO: 15 - DNA, 972 bp, Mouse
  CDS (17)..(556)
agccgccgccgccaccatggtgcaggcctggtatatggacgagtccaccgccgacccgcggaagccccaccgcgcacagcccgaccgccccgtgagcctggagcagctgcgcacgctcggagtgctctattggaagctagatgctgacaagtatgagaacgatccagaactagaaaagatccggaaaatgagaaactactcctggatggacatcatcaccatatgcaaagatacacttcccaattacgaggagaagatcaagatgttctttgaggaacatctgcatctggatgaggagatccgctacatcctggagggtagtgggtacttcgatgtcagggacaaggaggacaagtggatccggatttccatggagaagggggacatgattactcttcctgccggcatctatcaccgcttcacactggacgagaagaattacgtgaaggccatgcggctgtttgttggagaacctgtgtggacaccatacaaccggccagctgaccattttgatgcccgtgtacagtacatgagttttttggaaggaacagcatagcagtgctcctcaaagagaaaactgcactgtgtgaatctcctgctgtggtaaccgaatggaaagttgctcacttttctgcttttgtatttgaacttgaggctagactagctctctttgctaggattgtgagatcagtgtcttttaaatgaaagcctctctaaaagtgagttttacatggaagccacaaaaatgtgaaaaagtgaccttaattttccctaactgtcaagacttagaggtataggagccctggattggtatgtgcattcatgcatggccaatcttcatctcccagatctttaggtgtctgttggtgtgaagctatgcctcctgcaagagggcagttataaccagcacaactaaccagatgacgtttttctcctttgctgattgttgagtggggaagtggggttgttgtt

SEQ ID NO: 16 - protein, 179 aa, Mouse
MVQAWYMDESTADPRKPHRAQPDRPVSLEQLRTLGVLYWKLDADKYENDPELEKIRKMRNYSWMDIITICKDTLPNYEEKIKMFFEEHLHLDEEIRYILEGSGYFDVRDKEDKWIRISMEKGDMITLPAGIYHRFTLDEKNYVKAMRLFVGEPVWTPYNRPADHFDARVQYMSFLEGTA

SEQ ID NO: 17 - DNA, 706 bp, Zebrafish
  CDS (36)..(581): n at positions 634 and 642 is unknown
gtactgcgcatggagaccgaaccggactgttcaagatgagtgttttcgaggcatggtacatggatgaagagtccggagaggaccagagactcccgcacaaactgagcccgaatcagcccgtcagcgtccagcagctggagcacatcggagtctttcactggaagctgaacgctgatatctatgaaaatgaccccgaactgcagaagatccgagaggagaagggttattcctttatggacatcataaccattcacccggacaaactgcccgattaccaaaacaaactgaaaatgttttacgaagagcatctccacctggacgatgagatccgttatattctggaaggatcctcttattttgatgtgcgggacgaaggcgaccgctggatccgaatagcggtgtctaaaggcgacctcatcactttaccggccgggatttaccacagattcaccgtggacgaaagcaactacactaaagccatgcgtctgttcgtgggtgaacccgtctggaaggcctacaaccgtccagccgatgactttgacatccgcaaggaatacgtgaactcgctgggaagctcctgaaatgcctgatgggattgatttagtgctgagaatcagactctgcggtgccttanacagacangcagcaatagtagagctaacatgtcattacttagtcatcaagacacacctgatataaagattat

SEQ ID NO: 18 - protein, 181 aa, Zebrafish
  n at positions 634 and 642 is unknown
MSVFEAWYMDEESGEDQRLPHKLSPNQPVSVQQLEHIGVFHWKLNADIYENDPELQKIREEKGYSFMDIITIHPDKLPDYQNKLKMFYEEHLHLDDEIRYILEGSSYFDVRDEGDRWIRIAVSKGDLITLPAGIYHRFTVDESNYTKAMRLFVGEPVWKAYNRPADDFDIRKEYVNSLGSS